SEQUENCE LISTING (77 sequences)

SEQ ID NO: 1 - protein, 386 aa, Escherichia coli
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLAKRGCYRVLFVDQGDEQALRAALAEKPKLVLVESPSNPLLRVVDIAKICHLAREVGAVSVVDNTFLSPALQNPLALGADLVLHSCTKYLNGHSDVVAGVVIAKDPDVVTELAWWANNIGVTGGAFDSYLLLRGLRTLVPRMELAQRNAQAIVKYLQTQPLVKKLYHPSLPENQGHEIAARQQKGFGAMLSFELDGDEQTLRRFLGGLSLFTLAESLGGVESLISHAATMTHAGMAPEARAAAGISETLLRISTGIEDGEDLIADLENGFRAANKG

SEQ ID NO: 2 - DNA, 100 bp, Artificial Sequence
  Description of Artificial Sequence Synthetic oligonucleotide
tgacgtaggcctgataagcgtagcgcatcaggcgattccactccgcgccgctcttttttgctttagtattcccacgtctctgtaggctggagctgcttcg

SEQ ID NO: 3 - DNA, 100 bp, Artificial Sequence
  Description of Artificial Sequence Synthetic oligonucleotide
tatgcagctgacgacctttcgcccctgcctgcgcaatcacactcatttttaccccttgtttgcagcccggaagccattttcaggcaccagagtaaacatt

SEQ ID NO: 4 - DNA, 30 bp, Artificial Sequence
  Description of Artificial Sequence Synthetic primer
ggtacagaaaccagcaggctgaggatcagc

SEQ ID NO: 5 - DNA, 41 bp, Artificial Sequence
  Description of Artificial Sequence Synthetic primer
aaataacacttcacatcagccagactactgccaccaaattt

SEQ ID NO: 6 - DNA, 100 bp, Artificial Sequence
  Description of Artificial Sequence Synthetic oligonucleotide
ttcgtgtgccggacgagctacccgccgtcaatttcttgcgtgaagaaaacgtctttgtgatgacaacttctcgtgcgtcttgtaggctggagctgcttcg

SEQ ID NO: 7 - DNA, 100 bp, Artificial Sequence
  Description of Artificial Sequence Synthetic oligonucleotide
atccagcgttggattcatgtgccgtagatcgtatggcgtgatctggtagacgtaatagttgagccagttggtaaacagtacatatgaatatcctccttag

SEQ ID NO: 8 - DNA, 30 bp, Artificial Sequence
  Description of Artificial Sequence Synthetic primer
tcaccttcaacatgcaggctcgacattggc

SEQ ID NO: 9 - DNA, 30 bp, Artificial Sequence
  Description of Artificial Sequence Synthetic primer
ataaaaaaggcacccgaaggtgcctgaggt

SEQ ID NO: 10 - DNA, 99 bp, Artificial Sequence
  Description of Artificial Sequence Synthetic oligonucleotide
ctctacaatctgaaagatcacaacgagcaggtcagctttgcgcaagccgtaacccaggggttgggcaaaaatcaggggctgtaggctggagctgcttcg

SEQ ID NO: 11 - DNA, 99 bp, Artificial Sequence
  Description of Artificial Sequence Synthetic oligonucleotide
gattcatcatcaatttacgcaacgcagcaaaatcggcgggcagattatgtgaaagcaagggtaaatcagcacgttctgccatatgaatatcctccttag

SEQ ID NO: 12 - DNA, 22 bp, Artificial Sequence
  Description of Artificial Sequence Synthetic primer
cgctgaaccctaccgtgaacgg

SEQ ID NO: 13 - DNA, 24 bp, Artificial Sequence
  Description of Artificial Sequence Synthetic primer
gcgaccagaaccagggaaagtgcg

SEQ ID NO: 14 - DNA, 40 bp, Artificial Sequence
  Description of Artificial Sequence Synthetic primer
ttatcatgagagtgttgaagttcggcggtacatcagtggc

SEQ ID NO: 15 - DNA, 39 bp, Artificial Sequence
  Description of Artificial Sequence Synthetic primer
ttacccgggccgccgccccgagcacatcaaacccgacgc

SEQ ID NO: 16 - DNA, 24 bp, Artificial Sequence
  Description of Artificial Sequence Synthetic primer
ccgacagtaagacgggtaagcctg

SEQ ID NO: 17 - DNA, 22 bp, Artificial Sequence
  Description of Artificial Sequence Synthetic primer
agcttagtaaagccctcgctag

SEQ ID NO: 18 - DNA, 43 bp, Artificial Sequence
  Description of Artificial Sequence Synthetic primer
ccaatctgaataacatggcaatgtccagcgtttctggcccggg

SEQ ID NO: 19 - DNA, 43 bp, Artificial Sequence
  Description of Artificial Sequence Synthetic primer
cccgggccagaaacgctggacattgccatgttattcagattgg

SEQ ID NO: 20 - DNA, 34 bp, Artificial Sequence
  Description of Artificial Sequence Synthetic primer
tacgatgtacatggccttaatctggaaaactggc

SEQ ID NO: 21 - DNA, 34 bp, Artificial Sequence
  Description of Artificial Sequence Synthetic primer
tcccccgggttagttttccagtactcgtgcgccc

SEQ ID NO: 22 - DNA, 72 bp, Artificial Sequence
  Description of Artificial Sequence Synthetic primer
ccttttattcactaacaaatagctggtggaatatatgttgagctccgatgggagcctcactgttcatgccgg

SEQ ID NO: 23 - DNA, 53 bp, Artificial Sequence
  Description of Artificial Sequence Synthetic primer
aatcgcggatccgaatccggtcagatggcttcgagagcttgaagaatgtcagc

SEQ ID NO: 24 - DNA, 72 bp, Artificial Sequence
  Description of Artificial Sequence Synthetic primer
ccggcatgaacagtgaggctcccatcggagctcaacatatattccaccagctatttgttagtgaataaaagg

SEQ ID NO: 25 - DNA, 33 bp, Artificial Sequence
  Description of Artificial Sequence Synthetic primer
acgtcccgggcaagcccaaaggaagagtgaggc

SEQ ID NO: 26 - DNA, 98 bp, Artificial Sequence
  Description of Artificial Sequence Synthetic primer
ggcaaattttctggttatcttcagctatctggatgtctaaacgtataagcgtatgtagtgaggtaatcaggttatgccgattcgtgtgccggacgagc

SEQ ID NO: 27 - DNA, 99 bp, Artificial Sequence
  Description of Artificial Sequence Synthetic primer
cggaaataaaaaaggcacccgaaggtgcctgaggtaaggtgctgaatcgcttaacgatcgactatcacagaagattaatccagcgttggattcatgtgc

SEQ ID NO: 28 - DNA, 100 bp, Artificial Sequence
  Description of Artificial Sequence Synthetic oligonucleotide
caggcaccagagtaaacattgtgttaatggacgtcaatacatctggacatctaaacttctttgcgtatagattgagcaaacatatgaatatcctccttag

SEQ ID NO: 29 - DNA, 100 bp, Artificial Sequence; identical to SEQ ID NO: 2
  Description of Artificial Sequence Synthetic oligonucleotide

SEQ ID NO: 30 - DNA, 30 bp, Artificial Sequence; identical to SEQ ID NO: 4
  Description of Artificial Sequence Synthetic primer

SEQ ID NO: 31 - DNA, 30 bp, Artificial Sequence
  Description of Artificial Sequence Synthetic primer
ttcgtcgtcatttaacccgctacgcactgc

SEQ ID NO: 32 - DNA, 98 bp, Artificial Sequence; identical to SEQ ID NO: 26
  Description of Artificial Sequence Synthetic primer

SEQ ID NO: 33 - DNA, 99 bp, Artificial Sequence; identical to SEQ ID NO: 27
  Description of Artificial Sequence Synthetic primer

SEQ ID NO: 34 - DNA, 42 bp, Artificial Sequence
  Description of Artificial Sequence Synthetic primer
ttagacagaactgcagcgccgctccattcagccatgagatac

SEQ ID NO: 35 - DNA, 48 bp, Artificial Sequence
  Description of Artificial Sequence Synthetic primer
cgtaacgcccaagcttaaataacacttcacatcagccagactactgcc

SEQ ID NO: 36 - DNA, 102 bp, Artificial Sequence
  Description of Artificial Sequence Synthetic primer
tgctacagctggagctgttgacaattaatcatcggctcgtataatgtgtggaaggaggacagaccatgtcgcatactttaaaatcgaaaacgctccaagagc

SEQ ID NO: 37 - DNA, 48 bp, Artificial Sequence
  Description of Artificial Sequence Synthetic primer
cgtactgacgaccgggtcctaccagttggtaacttcttcggcctcacc

SEQ ID NO: 38 - DNA, 74 bp, Artificial Sequence
  Description of Artificial Sequence Synthetic primer
ggccggcgtgtagttgaacagtatcgaaatgagatggcatatattccaccagctatttgttagtgaataaaagg

SEQ ID NO: 39 - DNA, 74 bp, Artificial Sequence
  Description of Artificial Sequence Synthetic primer
ccttttattcactaacaaatagctggtggaatatatgccatctcatttcgatactgttcaactacacgccggcc

SEQ ID NO: 40 - DNA, 35 bp, Artificial Sequence
  Description of Artificial Sequence Synthetic primer
taatcgcggatccgcgtcatggtttttggccagcg

SEQ ID NO: 41 - DNA, 33 bp, Artificial Sequence; identical to SEQ ID NO: 25
  Description of Artificial Sequence Synthetic primer

SEQ ID NO: 42 - DNA, 51 bp, Artificial Sequence
  Description of Artificial Sequence Synthetic primer
gtcccccgggaatctagtctagattaaattacttcaagggcctgtttgagg

SEQ ID NO: 43 - DNA, 68 bp, Artificial Sequence
  Description of Artificial Sequence Synthetic primer
cacattttgttgcaaacttcacttctctttccatatattccaccagctatttgttagtgaataaaagg

SEQ ID NO: 44 - DNA, 68 bp, Artificial Sequence
  Description of Artificial Sequence Synthetic primer
ccttttattcactaacaaatagctggtggaatatatggaaagagaagtgaagtttgcaacaaaatgtg

SEQ ID NO: 45 - DNA, 33 bp, Artificial Sequence; identical to SEQ ID NO: 25
  Description of Artificial Sequence Synthetic primer

SEQ ID NO: 46 - DNA, 42 bp, Artificial Sequence
  Description of Artificial Sequence Synthetic primer
acgtggatccgaattccttattcgtcggcaagagcctgttgc

SEQ ID NO: 47 - DNA, 69 bp, Artificial Sequence
  Description of Artificial Sequence Synthetic primer
ggccgtacgggtccggtaaactgatcgatagccatatattccaccagctatttgttagtgaataaaagg

SEQ ID NO: 48 - DNA, 69 bp, Artificial Sequence
  Description of Artificial Sequence Synthetic primer
ccttttattcactaacaaatagctggtggaatatatggctatcgatcagtttaccggacccgtacggcc

SEQ ID NO: 49 - DNA, 33 bp, Artificial Sequence; identical to SEQ ID NO: 25
  Description of Artificial Sequence Synthetic primer

SEQ ID NO: 50 - DNA, 100 bp, Artificial Sequence
  Description of Artificial Sequence Synthetic oligonucleotide
ggctgactcgcaacccctgtccggtgctccggaaggtgccgaatatttaagagcagtgctgcgcgcgccggtttacgaggtgtaggctggagctgcttcg

SEQ ID NO: 51 - DNA, 99 bp, Artificial Sequence
  Description of Artificial Sequence Synthetic oligonucleotide
cctgaacgccgggttattggtttcgtcgtggcaatcgtagcccagctcattcagccgggtttcgaaatccggttcatggcatatgaatatcctccttag

SEQ ID NO: 52 - DNA, 24 bp, Artificial Sequence
  Description of Artificial Sequence Synthetic primer
gccccgaaccggtgcgtaaccgcg

SEQ ID NO: 53 - DNA, 21 bp, Artificial Sequence
  Description of Artificial Sequence Synthetic primer
ggtaagcgatgccgaactggc

SEQ ID NO: 54 - DNA, 99 bp, Artificial Sequence
  Description of Artificial Sequence Synthetic oligonucleotide
gctgacagcaatgtcagccgcagaccactttaatggccagtcctccgcgtgatgtttcgcggtatttatcgttcatatccatatgaatatcctccttag

SEQ ID NO: 55 - DNA, 99 bp, Artificial Sequence
  Description of Artificial Sequence Synthetic oligonucleotide
ggtaattaacgtaggtcgttatgagcactattcttcttccgaaaacgcagcacctggtagtctttcaggaagtcattagtgtaggctggagctgcttcg

SEQ ID NO: 56 - DNA, 25 bp, Artificial Sequence
  Description of Artificial Sequence Synthetic primer
gcgtctgcaatgacgcctttattcg

SEQ ID NO: 57 - DNA, 24 bp, Artificial Sequence
  Description of Artificial Sequence Synthetic primer
cgccataaaatatggttatccccg

SEQ ID NO: 58 - DNA, 42 bp, Artificial Sequence; identical to SEQ ID NO: 34
  Description of Artificial Sequence Synthetic primer

SEQ ID NO: 59 - DNA, 48 bp, Artificial Sequence; identical to SEQ ID NO: 35
  Description of Artificial Sequence Synthetic primer

SEQ ID NO: 60 - DNA, 42 bp, Artificial Sequence
  Description of Artificial Sequence Synthetic primer
cgcgcttctggtgccatgcccggatgtgccatggttgcggcg

SEQ ID NO: 61 - DNA, 40 bp, Artificial Sequence
  Description of Artificial Sequence Synthetic primer
cgccgcaaccatggcacatccgggcatggcaccagaagcg

SEQ ID NO: 62 - DNA, 46 bp, Artificial Sequence
  Description of Artificial Sequence Synthetic primer
gaacctcgagcgcatgattactcgcgtctgggcaacccaacgcgcg

SEQ ID NO: 63 - DNA, 46 bp, Artificial Sequence
  Description of Artificial Sequence Synthetic primer
cgcgcgttgggttgcccagacgcgagtaatcatgcgctcgaggttc

SEQ ID NO: 64 - DNA, 48 bp, Artificial Sequence
  Description of Artificial Sequence Synthetic primer
gaacctcgagcgcatgtgtactcgcgtcgcggcaacccaacgcgcgat

SEQ ID NO: 65 - DNA, 48 bp, Artificial Sequence
  Description of Artificial Sequence Synthetic primer
atcgcgcgttgggttgccgcgacgcgagtacacatgcgctcgaggttc

SEQ ID NO: 66 - protein, 562 aa, Bacillus anthracis
MAVSSFQCPTIFSSSSISGFQCRSDPDLVGSPVGGSSRRRVHASAGISSSAFTGDAGLSSRILRFPPNFVRQLSIKARRNCSNIGVAQIVAAKWSNNPSSALPSAAAAAATSSASAVSSAASAAAASSAAAPVAAAPPVVLKSVDEEVVVAEEGIREKIGSVQLTDSKHSFLSSDGSLTVHAGERLGRGIVTDAITTPVVNTSAYFFKKTAELIDFKEKRSVSFEYGRYGNPTTVVLEDKISALEGAESTLVMASGMCASTVMLLALVPAGGHIVTTTDCYRKTRIFMENFLPKLGITVTVIDPADIAGLEAAVNEFKVSLFFTESPTNPFLRCVDIELVSKICHKRGTLVCIDGTFATPLNQKALALGADLVVHSATKYIGHNDVLAGCICGSLKLVSEIRNLHHVLGGTLNPNAAYLIIRGMKTLHLRVQQQNSTAFRMAEILEAHPKVSHVYYPGLPSHPEHELAKRQMTGFGGVVSFEIDGDIETTIKFVDSLKIPYIAPSFGGCESIVDQPAIMSYWDLPQEERLKYGIKDNLVRFSFGVEDFEDVKADILQALEAI

SEQ ID NO: 67 - protein, 538 aa, Solanum tuberosum
MAVSSYARAFPSFECRSEPDFSGSLPHPKAGVRFSGKYNSGSNSRSQVYGLSSLIFRFPPNFVRQLSIKARRNCSNIGVAQVVAASWSNNQAGPEFTPAANAVDSASAAVTSIGITAGDEEVAVVENADCNDQNVQITGTGVKYASFLNSDGSVAIHAGERLGRGIVTDAITTPVVNTSAYFFNKTSDLIDFKEKRRASFEYGRYGNPTTVVLEEKISALEGAESTLIVASGMCASTVMFLALVPAGGHIVTTTDCYRKTRVFIETILPKMGITATVIDPADMGALELTLNQKKVDLFFTESPTNPFLRCVDIELVSKLCREKGALVCIDGTFATPLNQKALALGADLVVHSATKFLGHNDVLAGCISGPEKLVSVIRNLHHILGGALNPNAAYLIIRGMKTLHLRVQQQNSTALRMAEILEAHPKVKHVYYPGLPSHPEYHLAKKQMTGFGGVVSFEVDGDLLTTAKFVDALRIPYIAPSFGGCESIVDQPAIMSYWDLSQSDRAKYGILDNLVRFSFGVEDFEDVKADVLQALDSI

SEQ ID NO: 68 - protein, 535 aa, Francisella tularensis
MAVSSSHMRFTFECRSDPDFSPPPPSFDNLRRRNFRSSADSAGAAFHGISSLILRFPPNFQRQLSTKARRNCSNIGVAQIVAASWSNNSDNSPAAGAPAPPAATTDAATVPLPVVVAANEDVVVSAAADENGAVQLNSSSYSSFLKSDASKTIHAAERLGRGIETDGITTPVVNTSAYFFKKTADLIDFKENRQVSYEYGRYGNPTTVVLEEKISALEGAESTVIMASGMCASVVLFMALVPAGGHLVTTTDCYRKTRIFIETFLPKMGITTTVIDPADVGALESALEQHNVSLFFTESPTNPFLRCVDIKLVSELCHKKGTLLCIDGTFATPLNQKALALGADLILHSLTKYMGHHDVLGGCISGSIKVVSQIRTLHHVLGGTLNPNAAYLFIRGMKTLHLRVQQQNSTGMRMAKLLEAHPKVKRVYYPGLPSHPEHELAKRQMTGFGGVVSFEIDGDLHTTIKFIDSLKIPYIAASFGGCESIVDQPAILSYWDLPQSERAKYKIYDNLVRFSFGVEDFEDLKADVLQALEAI

SEQ ID NO: 69 - protein, 444 aa, Aspergillus terreus
AMAKAVDAAAAAAAIAPVDTTVVNEDVALVENETCNDQNVQFDSLPSMKYASFLNSDGSVAIHAGERLGRGIVTDAITTPVVNTSAYFFNKTSELIDFKEKRRASFEYGRYGNPTTVVLEEKISALEGAESTLLMASGMCASTVMLLALVPAGGHIVTTTDCYRKTRIFIETILPKMGITATVIDPADVGALELALNQKKVNLFFTESPTNPFLRCVDIELVSKLCHEKGALVCIDGTFATPLNQKALALGADLVLHSATKFLGHNDVLAGCISGPLKLVSEIRNLHHILGGALNPNAAYLIIRGMKTLHLRVQQQNSTALRMAEILEAHPKVRHVYYPGLQSHPEHHIAKKQMTGFGGAVSFEVDGDLLTTAKFVDALKIPYIAPSFGGCESIVDQPAIMSYWDLSQSDRAKYGIMDNLVRFSFGVEDFDDLKADILQALDSI

SEQ ID NO: 70 - protein, 508 aa, Zea mays
MATVSLTPQAVFSTSESGGALASATILRFPPNFVRQLSTKARRNCSNIGVAQIVAAAWSDCPAARPHLGGGGRRARGVASHAAAASAAAAASAAAEVSAIPNAKVAQPSAVVLAERNLLGSDASLAVHAGERLGRRIATDAITTPVVNTSAYWFNNSQELIDFKEGRHASFEYGRYGNPTTEALEKKMSALEKAESTVFVASGMYAAAAMLSALVPAGGHIVTTTDCYRKTRIYMETELPKRGISMTVIRPADMDALQNALDNNNVSLFFTETPTNPFLRCIDIEHVSNMCHSKGALLCIDSTFASPINQKALTLGADLVIHSATKYIAHNDVIGGCVSGRDELVSKVRIYHHVVGGVLNPNAAYLILRGMKTLHLRVQCQNNTALRMAQFLEEHLKIARVYYPGLPSHPEHHIAKSQMTGFGGVVSFEVAGDFDATRKFIDSVKIPYHAPSFGGCESIIDQPAIMSYWDSKEQRDIYGIKDNLIRFSIGVEDFEDLKNDLVQALEKI

SEQ ID NO: 71 - protein, 359 aa, Oryza sativa
SAYWFNNSQELIDFKEGRHASFEYGRYGNPTTEALEKKMSALEKAESTVFVASGMYASVAMLSALVPAGGHVVTTTDCYRKTRIYMETELPKTKSQMTVIRPADMDALQNALDNNNVSLFFTETPTNPFLRCIDIDLVSKMCHSKGALLCIDSTFASPINQKALTLGADLVIHSATKYIAHNDVIGGCISGRDELVSKVRIYHHVVGGVLNPNAAYLILRGMKTLHLRVQCQNNTAMRMAQFLEEHPKIARVYYPGLPSHPEHHIAKSQMTGFGGVISFEVAGDFDATRRFIDSVKIPYHAPSFGGCESIIDQPAIMSYWDSKEQREIYGIKDNLIRFSIGVEDFEDLKNDVVQALDKI

SEQ ID NO: 72 - protein, 397 aa, Ochrobactrum anthropi
MAIDQFTGPSTAAVHAGEPRQRAFDAITPPVVHSATYTFRDSAELIAFQSGDLEREEYGRYGNPTVRAVEARLAALESPTAPAAALLCASGMNALTTVMLALLPSGSHVILTDDGYRRTRQFVRTMLARLGVTHSVVAAADPAAIAAAIEPGRTRLIVTEAPTNPYLRVIDLAAVASIAREHRIKTLIDATFATPYNMRPLEYGIDLVVHSCSKYLAHNDLLAGVIIGRPPIISALRETQGILGGICDPHTAYLLGRGLKTFALRMERHNHNGQAVAEFLARHPRVSRVHYPGLPEHPDHAVARAQMRGFGGVVSFEVVGDLQSAMAVVDRLRLPYIAPSFGGVESLVEQPALMSYYELSSEERLAVGIRDNLIRLSCGIEDVSDLIADLQQALADE

SEQ ID NO: 73 - protein, 393 aa, Escherichia coli
MTKTWRPATQLVHGGTWRSEYGETSEAIYLTQGFVYDTSEPAEARFKGETDGFIYARYGSPTNDMFEKRMCALEGAEDARATASGMAAVAAAVLCQVKAGDHIVAARALFGSCRWVVETLAPKYGVECTLVDGRDLANWEKAIRPNTKVFFLESPTNPTLEVVDISGVAKLANQVGAKLIVDNVFATPLFQKPLELGAHIVVYSATKHIDQGACLGGVVLADKAWIDENLHDYFRHTGPAMSPFNAWTLLKGIETLPLRVRQQTENAAKIADFLAEQGKVAKVIYPGRKDHPQAEIIAKQMTGGSTLVCFELKGGKEAAFALQNALEIIKISNNLGDTKSLITHPATTTHKNLTEEARAELGISPGTVRLSAGIEDTDDLIEDFARGLTKVSA

SEQ ID NO: 74 - protein, 385 aa, Escherichia coli
MTRKQATIAVRSGLNDDEQYGCVVPPIHLSSTYNFTGFNEPRAHDYSRRGNPTRDVVQRALAELEGGAGAVLTNTGMSAIHLVTTVFLKPGDLLVAPHDCYGGSYRLFDSLAKRGCYRVLFVDQGDEQALRAALAEKPKLVLVESPSNPLLRVVDIAKICHLAREVGAVSVVDNTFLSPALQNPLALGADLVLHSCTKYLNHSDVVAGVVIAKDPDVVTELAWWANNIGVTGGAFDSYLLLRGLRTLVPRMELAQRNAQAIVKYLQTQPLVKKLYHPSLPENQGHEIAARQQKGFGAMLSFELDGDEQTLRRFLGGLSLFTLAESLGGVESLISHAATMTHAGMAPEARAAAGISETLLRISTGIEDGEDLIADLENGFRAANKG

SEQ ID NO: 75 - protein, 443 aa, Saccharomyces cerevisiae
MPSHFDTVQLHAGQENPGDNAHRSRAVPIYATTSYVFENSKHGSQLFGLEVPGYVYSRFQNPTSNVLEERIAALEGGAAALAVSSGQAAQTLAIQGLAHTGDNIVSTSYLYGGTYNQFKISFKRFGIEARFVEGDNPEEFEKVFDERTKAVYLETIGNPKYNVPDFEKIVAIAHKHGIPVVVDNTFGAGGYFCQPIKYGADIVTHSATKWIGHGTTIGGIIVDSGKFPWKDYPEKFPQFSQPAEGYHGTIYNEAYGNLAYIVHVRTELLRDLGPLMNPFASFLLLQGVETLSLRAERHGENALKLAKWLEQSPYVSWVSYPGLASHSHHENAKKYLSNGFGGVLSFGVKDLPNADKETDPFKLSGAQVVDNLKLASNLANVGDAKTLVIAPYFTTHKQLNDKEKLASGVTKDLIRVSVGIEFIDDIIADFQQSFETVFAGQKP

SEQ ID NO: 76 - protein, 456 aa, Burkholderia pseudomallei
MKDKNRNLNEKEGKENRGSQTNESQTKESREKKKSESLGVSTLAVHAGAKPDPATGARSVPIYQTAAYVFKDAEEAADLFGLRKEGNIYTRLMNPTTDVFEKRIAALEGGIGALAVASGMAAITTALLTFTRPGDEIISGDKLYGGTYELFNYTFPKLGRTVKFVDVGKPEEFKNAISEKTKAIYVESIGNPGLDIPDFEKLAEIAHGAGIPFVVDNTVSPLILRPIDHGVDIVVYSATKFIGHGTSIGGVIVDSGNFYWKPEKFPEICEPDPGYHGLKYKEAFGKAAFIAKARVQFIRDTGACISPFNSFLFTLGLETLPLRMKKHCDNALEIAKFLEKHPKVSWVSYPGLESHCSYELAKKYLKSGYGALIGFGIKGGARECKKFIEGLEIFSHLANIGDAKSLVIHPASTTHEQLSKEEQLACGVTEDFIRLSIGIEDAKDLISDIKQALSEV

SEQ ID NO: 77 - protein, 436 aa, Saccharomyces cerevisiae
MPKYDNSNADQWGFETRSIHAGQSVDAQTSARNLPIYQSTAFVFDSAEHAKQRFALEDLGPVYSRLTNPTVEALENRIASLEGGVHAVAFSSGQAATTNAILNLAGAGDHIVTSPRLYGGTETLFLITLNRLGIDVSFVENPDDPESWQAAVQPNTKAFFGETFANPQADVLDIPAVAEVAHRNSVPLIIDNTIATAALVRPLELGADVVVASLTKFYTNGSGLGGVLIDGGKFDWTVEKDGKPVFPYFVTPDAAYHGLKYADLGAPAFGLKVRVGLLRDTGSTLSAFNAWAAVQGIDTLSLRLERHNENAIKVAEFLNNHEKVEKVNFAGLKDSPWYATKEKLGLKYTGSVLTFEIKGGKDEAWAFIDALKLHSNLANIGDVRSLVVHPATTTHSQSDEAGLARAGVTQSTVRLSVGIETIDDIIADLEGGFAAI

(19) United States
(12) Patent Application Publication
(10) Pub. No.: US 2008/0286840 A1
(43) Pub. Date: Nov. 20, 2008

(54) Microorganisms Comprising Enzymes Express with Low Gamma-Elimination Activity

(76) Inventors: Rainer Figge, Riom (FR); Gwenaelle Bestel-Corre, Saint Beauzire (FR); Philippe Soucaille, Deyme (FR)

Correspondence Address: IP GROUP OF DLA PIPER US LLP, ONE LIBERTY PLACE, 1650 MARKET ST, SUITE 4900, PHILADELPHIA, PA, 19103, US

(21) Appl. No.: 11/883,903

(22) PCT Filed: Feb. 7, 2006
(86) PCT No.: PCT/EP2006/050726

§ 371 Date: Aug. 7, 2007

Related U.S. Application Data
(60) Provisional application No. 60/650,124, filed on Feb. 7, 2005.

(51) Int. Cl. C12P 13/04; C12N 1/00

(52) U.S. Cl. 435/106; 435/243

(57) ABSTRACT

A microorganism in which enzymes are expressed that have one or several of the following activities cystathionine-γ-synthases and/or phosphohomoserine sulfhydrylase and/or acylhomoserine sulfhydrylases and that have at the same time low γ-elimination activity is disclosed and also relates to recombinant enzymes that have one or several of the following activities cystathionine-γ-synthase and/or phosphohomoserine and/or acylhomoserine sulfhydrylase, have at the same time low γ-eliminase activity and are used for the fermentative production of amino acids, in particular, methionine.

### RELATED APPLICATION

This is a §371 of International Application No. PCT/EP2006/050726, with an international filing date of Feb. 7, 2006 (WO 2006/082254 A2, published Aug. 10, 2006), which claims priority of U.S. Provisional Application Ser. No. 60/650,124, filed Feb. 7, 2005.

This disclosure relates to microorganisms in which enzymes are expressed that have one or several of the following activities cystathionine-γ-synthases and/or phosphohomoserine sulfhydrylase and/or acylhomoserine sulfhydrylases and that have at the same time low γ-elimination activity. The disclosure also relates to recombinant enzymes that have one or several of the following activities cystathionine-γ-synthase and/or phosphohomoserine and/or acylhomoserine sulfhydrylase, have at the same time low γ-eliminase activity and are used for the fermentative production of amino acids, in particular methionine.

### BACKGROUND

Sulfur-containing compounds such as cysteine, homocysteine, methionine or S-adenosylmethionine are critical to cellular metabolism and are produced industrially to be used as food or feed additives and pharmaceuticals. In particular methionine, an essential amino acid, which cannot be synthesized by animals, plays an important role in many body functions. Aside from its role in protein biosynthesis, methionine is. involved in transmethylation and in the bioavailability of selenium and zinc. Methionine is also directly used as a treatment for medical disorders like allergy and rheumatic fever. Nevertheless most of the methionine that is produced is added to animal feed.

Chemically, D,L-methionine is commonly produced from acrolein, methyl mercaptan and hydrogen cyanide. However, the racemic mixture does not perform as well as pure L-methionine, e.g. in chicken feed additives (Saunderson, C. L., (1985) British Journal of Nutrition 54, 621-633). Pure L-methionine can be produced from racemic methionine e.g. through the acylase treatment of N-acetyl-D,L-methionine which increases production costs dramatically. The increasing demand for pure L-methionine coupled to environmental concerns renders microbial production of methionine attractive.

Only microorganisms and plants are capable of methionine biosynthesis. The pathway for L-methionine synthesis is well known in many microorganisms and plants (FIG. 1). Methionine in Escherichia coli is derived from the amino acid aspartate, but its synthesis requires the convergence of two additional pathways, cysteine biosynthesis and C1 metabolism (N-methyltetrahydrofolate). Aspartate is converted into homoserine by a sequence of three reactions. Homoserine can subsequently enter the threonine/isoleucine or methionine biosynthetic pathway. In E. coli entry into the methionine pathway requires the acylation of homoserine to succinyl-homoserine. This activation step allows subsequent condensation with cysteine, leading to the thioether-containing cystathionine, which is hydrolyzed to give homocysteine. The final methyl transfer leading to methionine is carried out by either a B₁₂-dependent or a B₁₂-independent methyltransferase.

Methionine biosynthesis in E. coli is regulated by repression and activation of methionine biosynthetic genes via the MetJ and MetR proteins. In addition to this transcriptional regulation, entry into the methionine specific pathway is tightly controlled by metA encoding homoserine transsuccinylase (EC 2.3.1.46). Aside from the transcriptional control of metA by MetJ and MetR, the enzyme is also feedback regulated by the major end-products of the pathway methionine and S-adenosylmethionine (Lee, L.-W et al. (1966) Multimetabolite control of a biosynthetic pathway by sequential metabolites, JBC 241 (22), 5479-5780). Feedback inhibition by these two products is synergistic, meaning that low concentrations of each metabolite alone are only slightly inhibitory, while in combination a strong inhibition is exerted.

Whereas in E. coli homoserine is activated to succinyl-homoserine that subsequently is transformed to γ-cystathionine by cystathionine-γ-synthase, gram-positive bacteria and spirochetes activate homoserine to acetyl-homoserine and are able to incorporate sulfur from H₂S by transforming acetyl-homoserine directly into homocysteine using acetyl-homoserine sulfhydrylase, called MetY in gram-positives and MetZ in spirochetes.

In contrast to methionine biosynthesis in eubacteria, in plants, the branch point between methionine and threonine/isoleucine biosynthesis lies at the level of phosphohomoserine. Homoserine is phosphorylated to yield phosphohomoserine, which can either react to threonine catalyzed by the enzyme threonine synthase or to γ-cystathionine and/or homocysteine using the plant enzyme METB having cystathionine-γ-synthase and phosphohomoserine sulfhydrylase activity. Recent data indicate that a similar pathway operates in archaea (White, 2003, The biosynthesis of cysteine and homocysteine in Methanococcus jannaschii, Biochim Biophys Acta. 1624(1-3):46-53), but the corresponding enzymes have not yet been characterized. Thus, in plants and most likely in some archaea the committed step for methionine production consists of the synthesis of γ-cystathionine from phosphohomoserine. In plants, methionine biosynthesis is controlled through the activity of the two key-enzymes cystathionine-γ-synthase (CGS) and threonine synthase (TS). CGS is regulated posttranscriptionally via an N-terminal regulatory region not found in the bacterial enzyme (Hacham et al. 2002 Plant Physiol. 128, 454-462). TS is feed-back activated by S-adenosyl methionine, a methionine derivative that signals high methionine concentrations. Both of these regulatory mechanisms direct the carbon flux away from methionine towards threonine, but are non-functional in prokaryotes.

Expression of different cystathionine-γ-synthases has been taught in WO 93/17112. Precise knowledge about cystathionine-γ-synthases was lacking at that time and sequences from plants were not known. JP2000-139471 describes the overexpression of E. coli cystathionine-γ-synthase, but the use of cystathionine-γ-synthase from different organisms has not been evaluated for the production of methionine in any organism.

MetB enzymes and their counterparts MetY/MetZ can accept a variety of different substrates, listed in Table 1. These enzymes can catalyze four different reactions, which all require activated homoserine. Activated homoserine can be either phospho-, acetyl or succinyl-homoserine. (i) Together with cysteine cystathionine-γ-synthase produces cystathionine, (ii) together with hydrogen sulfide sulfhydrylase synthesizes homocysteine, (iii) together with methylmercaptan methionine synthase produces methionine and (iiii) in the absence of a second substrate γ-eliminase catalyzes the dissociation of the substrate to α-ketobutyrate, ammonia and the activating group (acetate, succinate, phosphate). Several enzymes can catalyze more than one reaction, but with varying catalytic efficiencies. For example, E. coli MetB has the highest catalytic efficiency with cysteine and succinyl-homoserine as substrates and a relatively high γ-eliminase activity in the absence of cysteine (Aitken et al. 2003, Biochemistry 42, 11297-11306). Plant enzymes are equally good cystathionine producers, but have low γ-eliminase activity. In fact the k_{cat} of A. thaliana METB for the γ-eliminase activity is only 1/1500 of the E. coli enzyme. (Ravanel et al. 1998 Biochem. J. 331, 639-648). Some enzymes with low γ-elimination activity should favor high methionine production when introduced into E. coli.

TABLE 1
Preferred substrates for enzymes with cystathionine-γ-synthase and/or
phosphohomoserine sulfhydrylase and/or acylhomoserine sulfhydrylase activity from
different organisms.

| | Cystathionine-γ- | | Methionine | |
|---|---|---|---|---|
| Substrates | synthase | sulfhydrylase | synthase | γ-eliminase |
| Phosphohomo serine | Plants METB | plants | | plants METB |
| | archaea | | | |
| | Chloroflexus | | | |
| O-acetyl- | Bacillus metZ | Corynebacterium | | |
| homoserine | | metY | | |
| | | Spirochetes metZ | | |
| | | yeast MET25 | | |
| O-succinyl- | Escherichia metB | α-proteobacteria | Escherichia | Escherichia |
| homoserine | Xanthomonas | metZ | metB | metB |
| | metB | | | |

The type of activated homoserine and favored reaction are indicated for enzymes from different species or groups. For details see (Hacham et al. 2003, Mol. Biol. Evol. 20:1513-1520)

### SUMMARY

We provide a method of preparing an amino acid, its precursors or derivatives, comprising:
a) fermenting a microorganism producing the amino acid
b) concentrating the amino acid in cells of bacteria or in a medium and
c) isolating the desired amino acid/constituents of a fermentation broth and/or a biomass optionally remaining in portions or in a total amount (0-100%) in an end product,

wherein said microorganism expresses enzymes that have one or several of the following activities: cystathionine-γ-synthases and/or phosphohomoserine sulfhydrylase and/or acylhomoserine sulfhydrylases, and that have at the same time a γ-elimination activity that is at least two times inferior when compared to the E. coli cystathionine-γ-synthase.

We further provide a microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzymes are derived from one of the following species: Saccharomyces cerevisiae, Methanosarcina barkeri, and Chloroflexus aurantiacus.

We further provide a microorganism that expresses an enzyme having one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzyme has at least two of the following amino acids at positions 107E, 111Y, 165K, 403S.

We further provide a microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzymes are encoded by one of the following genes: metB from Saccharomyces cerevisiae, metY and/or metB from gram-positive bacteria.

We further provide a microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzyme has a proline at position 337 and/or an alanine at position 335.

Methionine production is enhanced when enzymes with cystathionine-γ-synthase and/or phosphohomoserine sulfhydrylase and/or acylhomoserine sulfhydrylase activity are expressed that have a low γ-elimination activity. Use of these enzymes reduces the production of α-ketobutyrate that can be transformed into isoleucine, which accumulates in the fermentation broth. We provide DNA fragments comprising genes encoding enzymes with cystathionine-γ-synthase and/or phosphohomoserine sulfhydrylase and/or acylhomoserine sulfhydrylase activity that have a low γ-eliminase activity. We also provide for the expression, especially overexpression of these enzymes in the production of methionine. We further provide microorganisms, preferentially enterobacteriaceae, coryneform bacteria or yeast, in which the aforementioned enzymes are expressed. In addition, we provide processes for the fermentative production of methionine its precursors or derivates using the microorganisms with the described properties.

### BRIEF DESCRIPTION OF THE DRAWINGS

FIG. 1. shows metabolic pathways converting oxaloacetate into threonine, isoleucine and methionine. MetB homologs can catalyze at least three reactions: synthesis of γ-cystathionine, sulfhydrylation or γ-elimination of activated homoserine. MetB homologs accepting preferentially phosphohomoserine are shown in red, succinylhomoserine accepting enzymes are represented in blue and acetylhomoserine transforming enzymes (MetB/metZ) are indicated in green.

FIG. 2. shows alignment of enzymes with cystathionine-γ-synthase and/or phosphohomoserine sulfhydrylase and/or acetylhomoserine sulfhydrylase activity of plants and bacterial species. The residues shown in grey boxes render the enzyme specific for the binding of phosphohomoserine. Highly conserved amino acids are shown in red, conserved residues in blue.

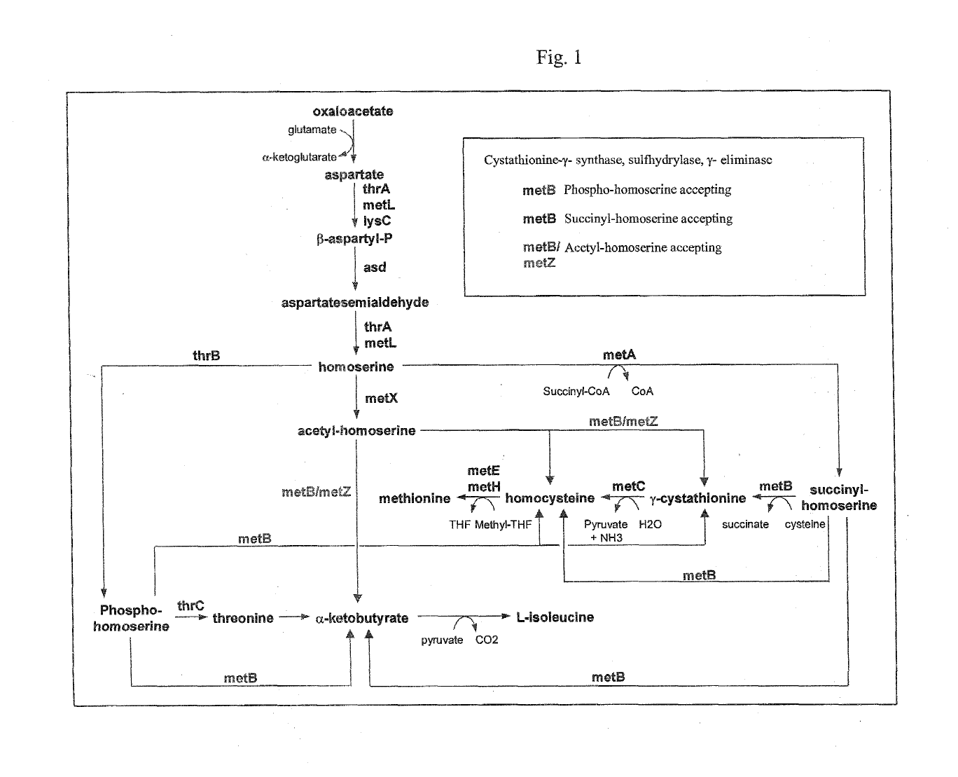

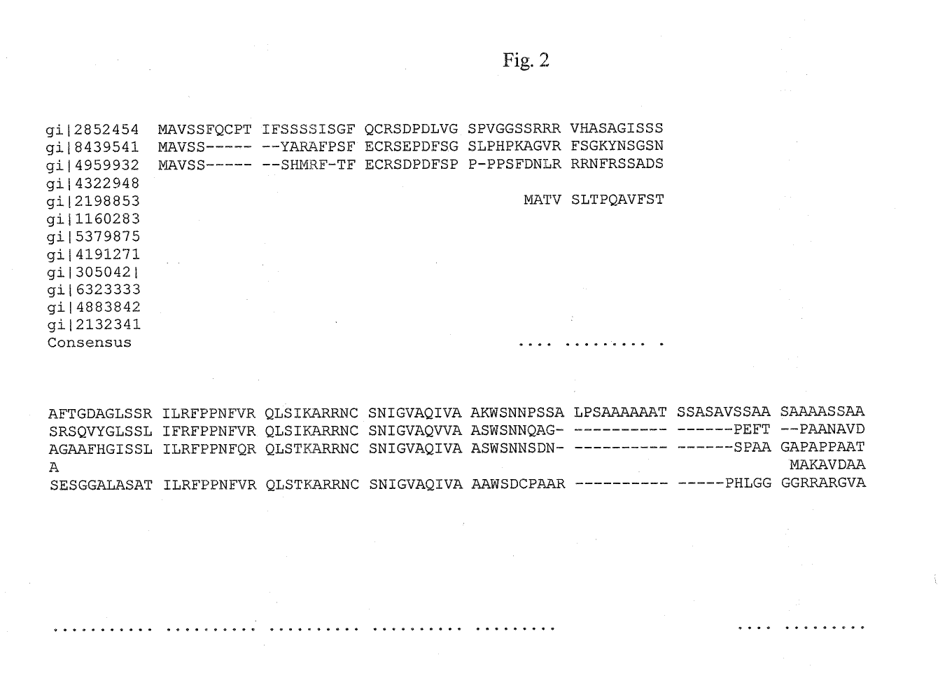

### DETAILED DESCRIPTION

Methionine is used in animal nutrition and pharmaceutical applications. Often a specific stereoisomer, in this case the biologically active L-form, is the preferred species. Since chemical synthesis can only provide racemic mixtures that are hard to resolve, it is of general interest to produce L-methionine by fermentation. It is thus advantageous to provide a strain by genetic engineering and improve—a fermentative process that yields L-methionine, its precursors or derivative in high quantities. In plants and microorganisms several enzymes are required for the fermentative production of methionine. Cystathionine-γ-synthase in E. coli (SEQ ID NO 1) is one of the key enzymes involved in methionine biosynthesis.

>E. coli |EG10582|MetB: 386 aa-Cystathionine
gamma-synthase
MTRKQATIAV RSGLNDDEQY GCVVPPIHLS STYNFTGFNE
PPAHDYSRRG NPTRDVVQRA LAELEGGAGA VLTNTGMSAI
HLVTTVFLKP GDLLVAPHDC YGGSYRLFDS LAKRGCYRVL
FVDQGDEQAL RAALAEKPKL VLVESPSNPL LRVVDIAKIC
HLAREVGAVS VVDNTFLSPA LQNPLALGAD LVLHSCTKYL
NGHSDVVAGV VIAKDPDVVT ELAWWANNIG VTGGAFDSYL
LLRGLRTLVP RMELAQRNAQ AIVKYLQTQP LVKKLYHPSL
PENQGHEIAA RQQKGFGAML SFELDGDEQT LRRFLGGLSL
FTLAESLGGV ESLISHAATM THAGMAPEAR AAAGISETLL
RISTGIEDGE DLIADLENGF RAANKG

In addition to its major activity, cystathionine γ-synthase has an undesired side-activity, succinyl-homoserine γ-eliminase, which transforms succinyl-homoserine into α-ketobutyrate, succinate and ammonia. This activity is especially pertinent if only low amounts of cysteine are present. In E. coli, it leads to a loss in methionine production, a problem that can be avoided by using cystathionine-γ-synthase that has a reduced γ-eliminase activity when compared to the E. coli enzyme.

We thus provide a microorganism for the fermentative production of amino acids in which one or several enzymes with cystathionine-γ-synthase and/or phosphohomoserine sulfhydrylase and/or acylhomoserine sulfhydrylase are expressed that have a low γ-eliminase activity.

Low γ-eliminase activity means preferentially a γ-eliminase activity lower than the activity observed with the native E. coli enzyme. In addition, it is desired that the enzymes with reduced γ-eliminase activity have specific cystathionine-γ-synthase and/or acylhomoserine sulfhydrylase and/or phosphohomoserine sulfhydrylase activities. The activity can eventually be lower than the activities of the native E. coli enzymes.

The expressed enzymes with cystathionine-γ-synthase and/or phosphohomoserine sulfhydrylase and/or acylhomoserine sulfhydrylase that have a low γ-eliminase activity are preferably different from the native enzymes present in the same organism. They may be either mutated genes of the same species or native or mutated genes of other species.

The microorganism may be transformed to introduce a gene coding for an enzyme with such low γ-eliminase activity.

Such gene may be introduced by different means available in the art:
modification of the native gene by homologous recombination to introduce mutations in the enzyme encoded by the gene to reduce γ-eliminase activity and keep the enzyme activity;
integrating into the genome of the microorganism a foreign gene coding for the selected enzyme known to have low γ-eliminase activity; the foreign gene being under control of regulatory elements functional in the host microorganism;
introducing a plasmid comprising a foreign gene coding for the selected enzyme known to have low γ-eliminase activity under control of regulatory elements functional in the host microorganism.

When the gene is integrated into the genome of the microorganism, it may advantageously be introduced in a locus selected to replace the native gene. Methods used to transform microorganisms are well known, including homologous recombination.

It is known that plant cystathionine-γ-synthases have lower γ-eliminase activities than the E. coli enzyme (Ravanel et al. 1998, Biochem. J. 331, 639-648). Nevertheless, it has never been shown that the use of plant enzymes in the fermentative production of methionine presents an advantage compared to the use of the native E. coli enzyme. We thus provide for the use of plant cystathionine-γ-synthase to increase the fermentative production of methionine.

Plant enzymes use phosphohomoserine as a substrate whereas the E. coli enzyme accepts succinyl-homoserine and to some extent acetyl-homoserine. Recently, it was shown that methionine biosynthesis in archaea probably proceeds via phosphohomoserine. So far, the enzymes have not been characterized. Thus, we also provide for the use of archaeal enzymes with cystathionine-γ-synthase and/or phosphohomoserine sulfhydrylase activity in the fermentative production of methionine.

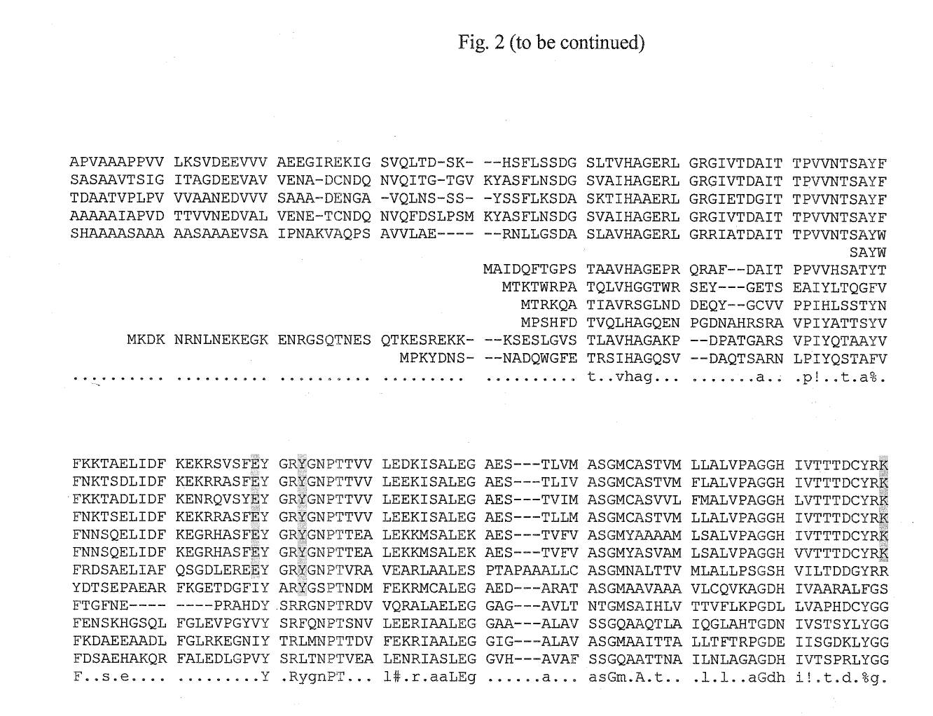

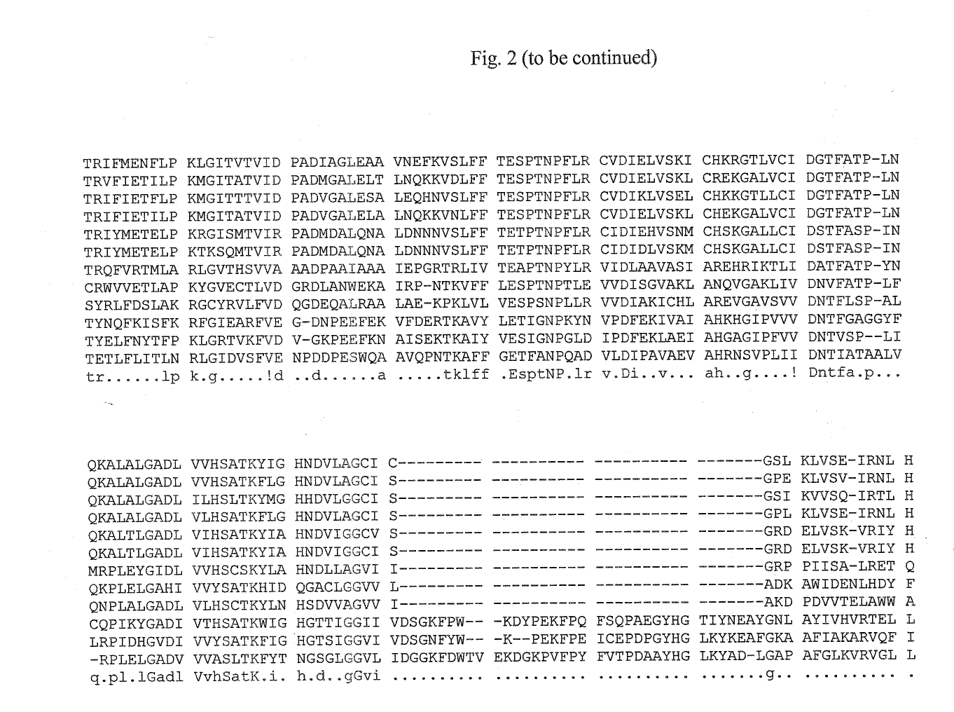

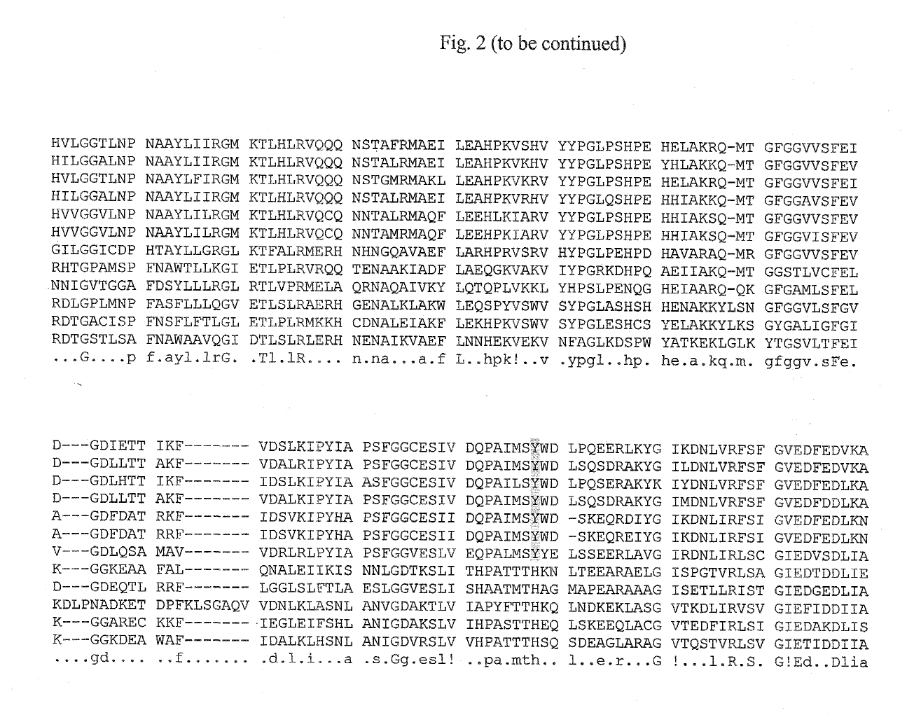

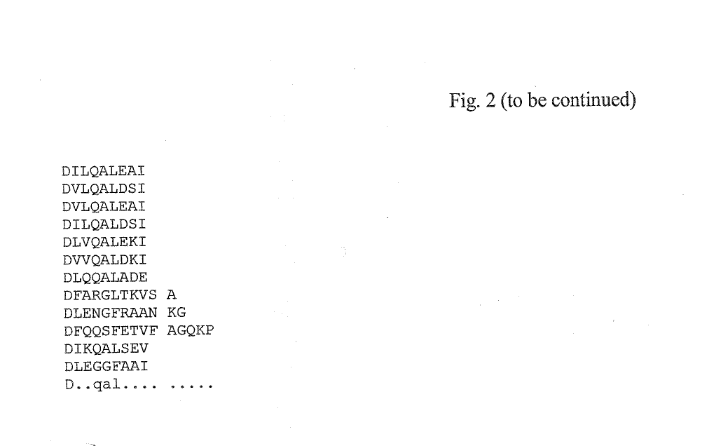

The closest homolog of plant cystathionine-γ-synthases in bacteria is found in the photosynthetic bacterium Chloroflexus. Alignments of this enzyme with plant enzymes demonstrate that the amino acids required for the binding of the phospho-group, which have been determined from structural comparisons between the E. coli and the plant enzymes (Steegborn et al. 2001 J. Mol Biol 311, 789-801) are conserved in the Chloroflexus enzyme (FIG. 2).

Thus, a microorganism expressing a homolog of the Chloroflexus gene, e.g. the gene >gi/53798754|ref|ZP_{—}00020132.2| COG0626 from Chloroflexus aurantiacus, is also provided.

Further provided are enzymes with cystathionine-γ-synthase/phosphohomoserine sulfhydrylase activity that have conserved the following amino acids at positions 107E, 111Y, 165K, 403S (see alignment, FIG. 2) permitting the use of phosphohomoserine as a substrate and thus exhibiting a reduced γ-elimination activity. Amino acid positions are given by reference to the sequence of Nicotiana tabacum. The same amino acid position can be identified without undue experimentation by simple sequence alignment (see FIG. 2).

As part of the biosynthetic pathway of threonine, phosphohomoserine is produced in E. coli and could thus be transformed directly into γ-cystathionine and/or homocysteine.

In contrast to plants and enterobacteriaceae, several gram-positive bacteria use a mechanism to activate homoserine that relies on the transfer of an acetyl group onto homoserine yielding acetyl-homoserine. Subsequently acetyl-homoserine is transformed into homocysteine by sulfhydrylation using an acetyl-homoserine sulfhydrylase MetY or into γ-cystathionine using cystathionine-γ-synthase. This reaction has been used to produce methionine by fermentation in coryneform bacteria as described in patent applications WO2004024933 and EP1313871. Nevertheless the corresponding genes have never been tested in E. coli and their γ-eliminase activity has not been determined.

Thus, we also provide for use of MetY enzymes in E. coli with low γ-eliminase activity, but comparable or increased sulfhydrylase activity with respect to the E. coli enzyme.

A decrease in the γ-eliminase activity of the native or introduced enzyme can also be obtained by selecting the enzyme, e.g. by directed evolution or site directed mutagenesis as described in Sambrook et al. (1989 Molecular cloning: a laboratory manual. 2^{nd} Ed. Cold Spring Harbor Lab., Cold Spring Harbor, N.Y.). The enzyme can also be selected through random mutagenesis using mutagens as NTG or EMS or by in vivo evolution as described in PCT/FR04/00354. Selected enzymes can also be synthetic genes that are based on natural genes and have selected codon usage and GC-content for the host organism. Therefore, the use of selected enzymes with reduced γ-eliminase activity or increased cystathionine-γ-synthase and/or phosphohomoserine sulfhydrylase and/or acylhomoserine sulfhydrylase activity is provided.

A particular example of an enzyme is cystathionine-γ-synthase and/or phosphohomoserine sulfhydrylase and/or acylhomoserine sulfhydrylase enzyme, in particular, the E. coli MetB enzyme, in which the alanine at position 337 has been replaced by a proline that is highly conserved in enzymes from different bacterial genera and/or comprising an alanine at position 335. Except as stated otherwise, positions are given by reference to the native E. coli enzyme.

We furthermore provide nucleotide sequences, DNA or RNA sequences, which encode cystathionine-γ-synthase and/or phosphohomoserine sulfhydrylase and/or acylhomoserine sulfhydrylase as defined above.

The cystathionine-γ-synthases and phosphohomoserine sulfhydrylases and/or acylhomoserine sulfhydrylases are advantageously selected among the enzymes corresponding to PFAM references PF01053 and to COG reference COG0626 and COG2873.

The PFAM (protein families database of alignments and hidden Markov models; http://www.sanfer.ac.uk/Software/Pfam/) form a large collection of alignments of protein sequences. Each PFAM makes it possible to visualize multiple alignments, see protein domains, evaluate distribution among organisms, gain access to other databases, and visualize known protein structures.

The COGs (clusters of orthologous groups of proteins; http://www.ncbi.nlm.nih.gov/COG/) are obtained by comparing the protein sequences from 43 fully sequenced genomes representing 30 major phylogenetic lines. Each COG is defined from at least three lines, which thus makes it possible to identify ancient conserved domains.

The preferred enzymes are the cystathionine-γ-synthase of Arabidopsis thaliana (accession number gi:1389725), the putative cystathionine-γ-synthase and/or sulfhydrylase of Methanosarcia barkeri (accession number gi:48839517), the acetyl-homoserine sulfhydrylase of Saccharomyces cerevisiae (accession number YLR303W), the putative cystathionine-γ-synthase and/or sulfhydrylase from Chloroflexus aurantiacus (gi:53798753) and the acetyl-homoserine sulfhydrylase from Corynebacterium glutamicum (gi:41324877) as synthetic gene adapted to E. coli. These sequences are aligned with the following representative sequences:

>gi|8439541|gb|AAF74981.1|AF082891_{—}1| cystathionine gamma-synthase isoform 1 [Solanum tuberosum]

>gi|4959932|gb|AAD34548.1|AF141602_{—}1| cystathionine-gamma-synthase precursor [Glycine max]

>gi/2198853|gb|AAB61348.1| cystathionine gamma-synthase [Zea mays]

>gi/4322948|gb|AAD16143.1| cystathionine gamma-synthase precursor [Nicotiana tabacum]

>gi|11602834|gb|AAG38873.1|AF076495_{—}1 cystathionine gamma-synthase [Oryza sativa]

>gi/305042|gb|AAB03071.1| cystathionine gamma-synthase [Escherichia coli]

The homologous sequences of these sequences that present a cystathionine-γ-synthase and/or phosphohomoserine sulfhydrylase and/or acylhomoserine sulfhydrylase activity, and present at least 80% homology, and preferably 90% homology, and more preferably 95% homology with the amino acid sequences described above are provided.

The means of identifying homologous sequences and their percentage homology are known and include, in particular, the BLAST program, and, in particular, the BLASTP program, which can be used from the web site http://www.ncbi.nlm.nih.gov/BLAST/ with the default parameters indicated on that site.

To determine which enzymes may be beneficial to methionine production when introduced into the corresponding strain, 7-eliminase activities may be evaluated enzymatically. For example, cystathionine-γ-synthase, 7-eliminase activity and sulfhydrylase activities can be determined in enzymatic tests with activated homoserine and (i) no other. substrate, or (ii) cysteine, or (iii) H₂S as substrate. The reaction is started by adding the protein extract containing the corresponding enzymatic activity, and the formation of homocysteine and/or γ-cystathionine is monitored by GC-MS after protein precipitation and derivatization with a silylating reagent. The gene(s) encoding cystathionine-γ-synthase and/or phosphohomoserine and/or acylhomoserine sulfhydrylase may be encoded chromosomally or extrachromosomally. Chromosomally there may be one or several copies on the genome that can be introduced by methods of recombination known in the field. Extrachromosomally genes may be carried by different types of plasmids that differ with respect to their origin of replication and, thus, their copy number in the cell. They may be present as 1-5 copies, ca 20 or up to 500 copies corresponding to low copy number plasmids with tight replication (pSC101, RK2), low copy number plasmids (pACYC, pRSF1010) or high copy number plasmids (pSK bluescript II).

The metB genes may be expressed using promoters with different strength that need or need not to be induced by inducer molecules. Examples are the promoters Ptrc, Ptac, Plac, the lambda promoter cI or other promoters known in the field.

Expression of the target genes may be boosted or reduced by elements stabilizing or destabilizing the corresponding messenger RNA (Carrier and Keasling (1998) Biotechnol. Prog. 15, 58-64) or the protein (e.g. GST tags, Amersham Biosciences)

We also provide microorganisms that contain one or several alleles encoding a cystathionine-γ-synthase and/or acylhomoserine and/or phosphohomoserine sulfhydrylase according to the invention.

Such strains are characterized by the fact that they possess a methionine metabolism which permits an increased flux towards methionine by either exclusively using phosphohomoserine accepting enzymes and thus reducing the amount of α-ketobutyrate produced or by using phosphohomoserine accepting enzymes in addition to acylhomoserine accepting enzymes thus increasing the flow towards methionine.

In particular, we provide for the preparation of L-methionine, its precursors or compounds derived thereof, by means of cultivating novel microorganisms and isolating the produced sulfur containing compounds.

An increase in the production of L-methionine, its precursors or compounds derived thereof, can be achieved by reducing the expression levels or deleting one of the following genes.

| Gene | Genbank entry | activity |
|---|---|---|
| ackA | 1788633 | acetate kinase |
| pta | 1788635 | phosphotransacetylase |
| acs | 1790505 | acetate synthase |
| aceE | 1786304 | pyruvate deydrogenase E1 |
| aceF | 1786305 | pyruvate deydrogenase E2 |
| lpd | 1786307 | pyruvate deydrogenase E3 |
| sucC | 1786948 | succinyl-CoA synthetase, beta subunit |
| sucD | 1786949 | succinyl-CoA synthetase, alpha subunit |
| pck | 1789807 | phosphoenolpyruvate carboxykinase |
| pykA | 1788160 | pyruvate kinase II |
| pykF | 1787965 | pyruvate kinase I |
| poxB | 1787096 | pyruvate oxidase |
| ilvB | 1790104 | acetohydroxy acid synthase I, large subunit |
| ilvN | 1790103 | acetohydroxy acid synthase I, small subunit |
| ilvG | 1790202 | acetohydroxy acid synthase II, large subunit |
| | 1790203 | |
| ilvM | 1790204 | acetohydroxy acid synthase II, small subunit |
| ilvI | 1786265 | acetohydroxy acid synthase III, large subunit |
| ilvH | 1786266 | acetohydroxy acid synthase III, small subunit |
| aroF | 1788953 | DAHP synthetase |
| aroG | 1786969 | DAHP synthetase |
| aroH | 1787996 | DAHP synthetase |

An additional increase in the production of L-methionine, its precursors or compounds derived thereof can be achieved by overexpressing one or several of the following genes: the pyruvate carboxylase from Rhizobium etli (pyc, U51439), or one of its homologs, the homoserine synthesizing enzymes encoded by the genes thrA (homoserine dehydrogenase/aspartokinase, 1786183), preferably with reduced feed-back sensitivity, metL (homoserine dehydrogenase/aspartokinase, g1790376) or lysC (aspartokinase, 1790455) and asd (aspartate semialdehyde dehydrogenase, 1789841) or a combination thereof.

A further increase in the production of L-methionine, its precursors or compounds derived thereof, is possible by overexpressing genes involved in sulfate assimilation and production of cysteine. An increased amount of sulfur containing compounds will equally reduce the γ-eliminase activity. This can be achieved by overexpressing the following genes (see below) or by deregulating the pathway through the introduction of a constitutive cysB allele as described by Coyler and Kredich (1994 Mol Microbiol 13 797-805) and by introducing a cysE allele encoding a serine acetyl transferase with decreased sensitivity for its inhibitor L-cysteine (U.S. Pat. No. 6,218,168; Denk & Bock 1987 J Gen Microbiol 133 515-25). The following genes need to be overexpressed.

| | | |
|---|---|---|
| CysA | 1788761 | sulfate permease |
| CysU | 1788764 | cysteine transport system |
| CysW | 1788762 | membrans bound sulphate transport system |
| CysZ | 1788753 | ORF upstream of cysK |
| cysN | 1789108 | ATP sulfurylase |
| cysD | 1789109 | sulfate adenylyltransferase |
| cysC | 1789107 | adenylylsulfate kinase |
| cysH | 1789121 | adenylylsulfate reductase |
| cysI | 1789122 | sulfite reductase, alpha subunit |
| cysJ | 1789123 | sulfite reductase, beta subunit |
| cysE | 1790035 | serine acetyltransferase |
| cysK | 1788754 | cysteine synthase |
| cysM | 2367138 | O-acetyl-sulfhydrolase |
| cysW | 1788762 | sulfate transport |
| cysT | | sulfate transport |
| cysZ | 1788753 | sulfate transport |
| sbp | 1790351 | Periplasmic sulfate-binding protein |

In addition, genes involved in the production of C1 (methyl) groups may be enhanced by overexpressing the following genes:

| | | |
|---|---|---|
| serA | 1789279 | phosphoglycerate dehydrogenase, preferably feed- |
| | | back resistant |
| serB | 1790849 | phosphoserine phosphatase |
| serC | 1787136 | phosphoserine aminotransferase |
| glyA | 1788902 | serine hydroxymethyltransferase |
| metF | 1790377 | 5,10-Methylenetetrahydrofolate reductase |

In addition, genes directly involved in the production of methionine may be overexpressed:

| | | |
|---|---|---|
| metB | 1790375 | Cystathionine-γ-synthase |
| metC | 1789383 | Cystathionine-β-lyase |
| metH | 1790450 | B12-dependent homocysteine-N5- |
| | | methyltetrahydrofolate transmethylase |
| metE | 2367304 | Tetrahydropteroyltriglutamate methyltransferase |
| metF | 1790377 | 5,10-Methylenetetrahydrofolate reductase |
| metR | 1790262 | Positive regulatory gene for metE and metH and |
| | | autogenous regulation |

Furthermore, expression of genes in pathways degrading methionine or deviating from the methionine production pathway may be reduced or the genes may be deleted.

| | | |
|---|---|---|
| speD | 1786311 | S-Adenosylmethionine decarboxylase |
| speC | 1789337 | Ornithine decarboxylase |
| astA | 1788043 | Arginine succinyltransferase |
| dapA | 1788823 | Dihydrodipicolinate synthase |

Anaplerotic reactions may be boosted by expressing

| | | |
|---|---|---|
| ppc | 1790393 | phosphoenolpyruvate carboxylase |
| pps | 1787994 | phosphoenolpyruvate synthase |

A further increase in the production of L-methionine, its precursors or compounds derived thereof, is achieved by deleting the gene for the repressor protein MetJ, responsible for the down-regulation of the methionine regulon as suggested in JP 2000157267-A/3 (see also GenBank 1790373).

Production of methionine may be further increased by using an altered metB allele that uses preferentially or exclusively H₂S and thus produces homocysteine from O-succinyl-homoserine as has been described in PCT No PCT/FR04/00354, the content of which is incorporated herein by reference.

The organism may be either E. coli or C. glutamicum or Saccharomyces cerevisiae.

We also provide a process for the production of L-methionine, its precursors or compounds derived thereof, which is/are usually prepared by fermentation of the designed bacterial strain.

The terms ‘culture’ and ‘fermentation’ are used indifferently to denote the growth of a microorganism on an appropriate culture medium containing a simple carbon source.

A simple carbon source is a source of carbon that can be used by those skilled in the art to obtain normal growth of a microorganism, in particular, of a bacterium. It can be an assimilable sugar such as glucose, galactose, sucrose, lactose or molasses, or by-products of these sugars. An especially preferred simple carbon source is glucose. Another preferred simple carbon source is sucrose.

Those skilled in the art are able to define the culture conditions for the microorganisms. In particular, the bacteria are fermented at a temperature between 20° C. and 55° C., preferentially between 25° C. and 40° C., and more specifically about 30° C. for C. glutamicum and about 37° C. for E. coli.

The fermentation is generally conducted in fermenters with an inorganic culture medium of known defined composition adapted to the bacteria used, containing at least one simple carbon source and, if necessary, a co-substrate necessary for the production of the metabolite.

In particular, the inorganic culture medium for E. coli can be of identical or similar composition to an M9 medium (Anderson, 1946, Proc. Natl. Acad. Sci. USA 32:120-128), an M63 medium (Miller, 1992; A Short Course in Bacterial Genetics: A Laboratory Manual and Handbook for Escherichia coli and Related Bacteria, Cold Spring Harbor Laboratory Press, Cold Spring Harbor, N.Y.) or a medium such as defined by Schaefer et al. (1999, Anal. Biochem. 270: 88-96).

Analogously, the inorganic culture medium for C. glutamicum can be of identical or similar composition to BMCG medium (Liebl et al., 1989, Appl. Microbiol. Biotechnol. 32: 205-210) or to a medium such as that described by Riedel et al. (2001, J. Mol. Microbiol. Biotechnol. 3: 573-583). The media can be supplemented to compensate for auxotrophies introduced by mutations.

After fermentation, L-methionine, its precursors or compounds derived thereof, is/are recovered and purified if necessary. The methods for the recovery and purification of the produced compound such as methionine in the culture media are well known. The sulfur source used for the fermentative production of L-methionine, its precursors or compounds derived thereof, may be any of the following:, sulfate, thiosulfate, hydrogen sulfide, methylmercaptam.

### EXAMPLE 1

### Construction of a Strain Using Exclusively Plant Phosphohomoserine Accepting METB with Low γ-Eliminase Activity for Methionine Production

To verify that plant CGS have a lower γ-eliminase activity than the E. coli enzyme, ratios between γ-eliminase activity and CGS activity of the Arabidopsis METB enzyme and the E. coli enzyme were determined in crude extracts. For this purpose, E. coli strains were constructed in which the chromosomal copy of the metB gene was deleted and either E. coli or Arabidopsis CGS was expressed from a plasmid. Concomitantly with the metB deletion the metJ gene was also eliminated (see below).

E. coli ΔmetJB strains carrying either wild-type or heterologous acylhomoserine or phosphohomoserine accepting cystathionine-γ-synthase and/or sulfhydrylases were cultured in minimal medium with 5 g/l glucose and harvested at late log phase. Cells were resuspended in cold potassium phosphate buffer and sonicated on ice (Branson sonifier, 70W). After centrifugation, proteins contained in the supernatants were quantified (Bradford, 1976).

Ten μl of the extracts were incubated for 10 minutes at 30° C. with either 5 mM O-succinyl-homoserine or O-acetyl-homoserine and either 1.5 mM sodium sulfide or cysteine. Proteins were precipitated with acetone and homocysteine or γ-cystathionine produced during the enzymatic reaction was quantified by GC-MS, after derivatization with tert-butyldimethylsilyltrifluoroacetamide (TBDMSTFA). L-Serine [1-13C] was included as an internal standard. Alternatively, the disappearance of cysteine was measured using the same protocol.

To inactivate the metB and metJ gene, the homologous recombination strategy described by Datsenko & Wanner (2000) was used. This strategy allowed the insertion of a chloramphenicol resistance cassette, while deleting most of the genes concerned. For this purpose the following oligonucleotides were used:

DmetJR (SEQ ID NO 2):
tgacgtaggc ctgataagcg tagcgcatca ggcgattcca
ctccgcgccg ctcttttttg ctttagtatt cccacgtctc
TGTAGGCTGG AGCTGCTTCG
with
a region (lower case) homologous to the sequence
(4125596-4125675) of the gene metJ (reference
sequence on the website http://genolist.pasteur.fr
/Colibri/),
a region (upper case) for the amplification of the
chloramphenicol resistance cassette (reference
sequence in Datsenko, K.A. & Wanner, B.L., 2000,
PNAS, 97: 6640-6645),
DmetJBF (SEQ ID NO 3):
tatgcagctg acgacctttc gcccctgcct gcgcaatcac
actcattttt accccttglt tgcagcccgg aagccatttt
CAGGCACCAG AGTAAACATT
with
a region (lower case) homologous to the sequence
(4127460-4127381) of the gene metB and a region
(4126116-4126197) homologous to the promoter of
metL
a region (upper case) for the amplification of the
chloramphenicol resistance cassette.

The oligonucleotides DmetJBR and DmetJBF were used to amplify the chloramphenicol resistance cassette from the plasmid pKD3. The PCR product obtained was then introduced by electroporation into the strain MG1655 (pKD46) in which the Red recombinase enzyme was expressed permitting the homologous recombination. Chloramphenicol resistant transformants were selected and the insertion of the resistance cassette was verified by a PCR analysis with the oligonucleotides MetJR and MetJF defined below. The strain retained was designated MG1655 (ΔmetJB::Cm).

MetJR (SEQ ID NO 4):
ggtacagaaa ccagcaggct gaggatcagc
(homologous to the sequence from 4125431 to
4125460).
MetLR (SEQ ID NO 5):
aaataacact tcacatcagc cagactactgc caccaaattt
(homologous to the sequence from 4127500 to
4157460).

The chloramphenicol resistance cassette was then eliminated. The plasmid pCP20 carrying recombinase FLP acting at the FRT sites of the chloramphenicol resistance cassette was introduced into the recombinant strains by electroporation. After a series of cultures at 42° C., the loss of the chloramphenicol resistance cassette was verified by a PCR analysis with the same oligonucleotides as those used previously.

For the production of methionine using phosphohomoserine-accepting METB from Arabidopsis thaliana, the following Escherichia coli strain was constructed. The methionine repressor metJ together with E. coli metB were deleted as described above. Subsequently, the gene metA encoding succinyl-homoserine transferase and/or the gene thrC encoding threonine synthase were deleted. The deletion strategy has been exemplified for the metJB deletion. The deletion of all other genes was based on the same strategy using the oligonucleotides indicated. The oligonucleotides for the deletions of the metA and thrC genes are indicated below. The numbers in parentheses indicate the regions that are homologous to the E. coli chromosome. Oligonucleotides beginning with D were used for the actual deletion, other oligonucleotides for the verification of the constructs or for specific purposes as indicated.

For deletion of the metA gene, the following oligonucleotides were used:

DmetAF (4211866-4211945; SEQ ID NO 6):
ttcglgtgcc ggacgagcta cccgccgtca atttcttgcg
tgaagaaaac gtctttgtga tgacaacttc tcgtgcgtct
TGTAGGCTGG AGCTGCTTCG
DmetAR (4212785-4212706; SEQ ID NO 7):
atccagcgtt ggattcatgt gccgtagatc gtatggcgtg
atctggtaga cgtaatagtt gagccagttg gtaaacagta
CATATGAATA TCCTCCTTAG
MetAF (4211759-4211788; SEQ ID NO 8):
tcaccttcaa catgcaggct cgacattggc
MetAR (4212857-4212828; SEQ ID NO 9):
ataaaaaagg cacccgaagg tgcctgaggt

For deletion of the thrC gene, the following oligonucleotides were used:

DthrCF (3740-3821; SEQ ID NO 10):
ctctacaatc tgaaagatca caacgagcag gtcagctttg
cgcaagccgt aacccagggg ttgggcaaaa atcaggggcT
GTAGGCTGGAG CTGCTTCG
DthrCR (5012-4932; SEQ ID NO 11):
gattcatcat caatttacgca acgcagcaaa atcggcgggc
agattatgtg aaagcaaggg taaatcagca cgttctgcCA
TATGAATATC CTCCTTAG
thrCF (3490-3511; SEQ ID NO 12):
cgctgaaccc taccgtgaac gg
thrCR (5284-5260; SEQ ID NO 13):
gcgaccagaa ccagggaaag tgcg

To further boost the production of homoserine, the aspartokinase/homoserine a thrA* allele with reduced feed-back resistance to threonine was expressed from the plasmid pCL1920 (Lerner & Inouye, 1990, NAR 18, 15 p 4631) using the promoter Ptrc. For the construction of plasmid pME101-thrA*1 thrA was PCR amplified from genomic DNA using the following oligonucleotides:

BspH1thrA (SEQ ID NO 14):
ttaTCATGAgagtgttgaagttcggcggtacatcagtggc
Sma1thrA (SEQ ID NO 15):
ttaCCCGGGccgccgccccgagcacatcaaacccgacgc

The PCR amplified fragment was cut with the restriction enzymes BspHI and SmaI and cloned into the NcoI/SmaI sites of the vector pTRC99A (Stratagene). For the expression from a low copy vector the plasmid pME101 was constructed as follows. The plasmid pCL1920 was PCR amplified using the oligonucleotides PME101F and PME101R and the BstZ171-XmnI fragment from the vector pTRC99A harboring the lacI gene and the Ptrc promoter was inserted into the amplified vector. The resulting vector and the vector harboring the thrA gene were restricted by ApaI and SmaI and the thrA containing fragment was cloned into the vector pME101. To relieve ThrA from feed-back inhibition the mutation F318S was introduced by site-directed mutagenesis (Stratagene) using the oligonucleotides ThrAF F318S and ThrAR F318S, resulting in the vector pME101-thrA*1, called pSB1.

PME101F (SEQ ID NO 16):
Ccgacagtaagacgggtaagcctg
PME101R (SEQ ID NO 17):
Agcttagtaaagccctcgctag
ThrAF F318S (SmaI) (SEQ ID NO 18):
Ccaatctgaataacatggcaatgtccagcgtttctggcccggg
ThrAR F318S (SmaI) (SEQ ID NO 19):
Cccgggccagaaacgctggacattgccatgttattcagattgg

To transform part of the homoserine produced into phosphohomoserine the thrB gene was cloned into the vector pSB1. thrB was PCR amplified using the oligonucleotides thrA′BF and thrA′BR

thrA′BF (SEQ ID NO 20):
tacgatgtac atggccttaa tctggaaaac tggc
thrA′BR (SEQ ID NO 21):
tcccccgggT TAGTTTTCCA GTACTCGTGC GCCC

The PCR fragment was digested with BsrG1 and SmaI and cloned into the vector pSB1 cut by the same restriction enzymes resulting in plasmid pSB2.

Subsequently, the enzyme METB from Arabidopsis thaliana was PCR amplified from a cDNA clone that is commercially available (TAIR, http://arabidopsis.org/contact/) using the oligonucleotides gapA-cgsAF and cgsAR. The oligonucleotides gapA-cgsAF consisted of nucleotides 1 to 38 of METB (fat) and nucleotides 1860761 to 1960799 (underlined) of E. coli pertaining to the GapA promoter region.

gapA-cgsAF (SEQ ID NO 22):
ccttttattc actaacaaat agctggtgga atatatgttg
agctccgatg ggagcctcac tgttcatgcc gg
cgsAR (SEQ ID NO 23):
AATCGCGGAT CCGAATCCGG TCAGATGGCT TCGAGAGCTT
GAAGAATGTC AGC

At the same time the GapA promoter region of the E. coli gene GapA was amplified using the oligonucleotides gapA-cgsAR and GapAF. The oligonucleotide gapA-cgsAR harbors base 1 to 39 of the MetB gene and bases 1860799 to 1860761 from the E. coli chromosome corresponding to the GapA promoter region. The oligonucleotide GapAF corresponds to bases 1860639 to 1860661 of the E. coli genome.

gapA-cgsAR (SEQ ID NO 24):
ccggcatgaa cagtgaggct cccatcggag ctcaacatat
attccaccag ctatttgtta gtgaataaaag g
GapAF (SEQ ID NO 25):
acgtcccggg caagcccaaa ggaagagtga ggc

Both fragments were subsequently fused using the oligonucleotides cgsAR and GapAF (Horton et al. 1989 Gene 77:61-68), cut with the restriction enzymes BamHI and SmaI and cloned into the corresponding restriction sites of vectors pSB1 and pSB2 giving vector pSB3 (pME101thrA*-metBAt) and pSB4 (pME101thrA*-thrB-metBAt), respectively.

The vectors pSB3 and pSB4 were subsequently introduced into the strain ΔmetBJ ΔmetA ΔthrC.

### EXAMPLE 2

### Construction of a Strain Using Exclusively Plant METB with Low γ-Eliminase Activity for Methionine Production Via O-Succinylhomoserine

For the use of plant METB for the production of methionine via succinyl homoserine, the strain ΔmetBJ metA* was constructed. Construction was initiated with the strains described in Example 1 in which a feed-back resistant homoserine transsuccinylase metA*11 (described in patent application PCT IB2004/001901) was introduced into the genome. For this purpose the metA*11 allele was amplified from the E. coli chromosome using the following oligonucleotides.

MetArcF (4211786-4211883; SEQ ID NO 26):
ggcaaatttt ctggttatct tcagctatct ggatgtctaa
acgtataagc gtatgtagtg aggtaatcag gttatgccga
ttcgtgtgcc ggacgagc
MetArcR (4212862-4212764; SEQ ID NO 27):
cggaaataaa aaaggcaccc gaaggtgcct gaggtaaggt
gctgaatcgc ttaacgatcg actatcacag aagattaatc
cagcgttgga ttcatgtgc

The plasmid pKD46 was introduced into strains MG1655 ΔmetA ΔthrC and MG1655 ΔmetA and transformed with the DNA fragment harboring the metA*11 allele. Clones were selected for methionine prototrophy on modified M9 plates. Subsequently the mutation ΔmetBJ was added as described above. The plasmid pSB3 and pSB4 were introduced into the two strains.

### EXAMPLE 3

### Construction of a Strain Using Plant Phosphohomoserine Accepting METB with Low γ-Eliminase Activity and Simultaneous Use of E. coli MetB

To further boost methionine synthesis, a strain was constructed that simultaneously produces methionine via O-succinyl homoserine and phosphohomoserine. The construction was initiated with the strains ΔmetA and ΔmetA ΔthrC described in Example 1 in which the gene metJ was deleted using the following oligonucleotides:

DmetJF with 100 bases (SEQ ID NO 28):
caggcaccag agtaaacatt gtgttaatgg acgtcaatac
atctggacat ctaaacttct ttgcgtatag attgagcaaa
CATATGAATA TCCTCCTTAG
DmetJR with 100 bases (SEQ ID NO 29):
tgacgtaggc ctgataagcg tagcgcatca ggcgattcca
ctccgcgccg ctcttttttg ctttagtatt cccacgtctc
TGTAGGCTGG AGCTGCTTCG
MetJR (SEQ ID NO 30):
ggtacagaaa ccagcaggct gaggatcagc
(homologous to the sequence from 4125431 to
4125460).
MetBR (SEQ ID NO 31):
ttcgtcgtca tttaacccgc tacgcactgc
(homologous to the sequence from 4126305 to
4126276).

Subsequently, a feed-back resistant homoserine transsuccinylase metA*11 (described in patent application PCT IB2004/001901) was introduced into the genome. For this purpose, the metA*11 allele was amplified from the E. coli chromosome using the following oligonucleotides.

MetArcF (4211786-4211883; SEQ ID NO 32):
ggcaaatttt ctggttatct tcagctatct ggatgtctaa
acgtataagc gtatgtagtg aggtaatcag gttatgccga
ttcgtgtgcc ggacgagc
MetArcR (4212862-4212764; SEQ ID NO 33):
cggaaataaa aaaggcaccc gaaggtgcct gaggtaaggt
gctgaatcgc ttaacgatcg actatcacag aagattaatc
cagcgttgga ttcatgtgc

The plasmid pKD46 was introduced into strains MG1655 ΔmetJ ΔmetA ΔthrC and MG1655 ΔmetJ ΔmetA that were transformed with the DNA fragment harboring the metA*11 allele. Clones were selected for methionine prototrophy on modified M9 plates (supplemented with threonine if necessary).

Similarly to the construction of pSB3 and pSB4 using metBAt in Example 1, the metB gene of E. coli with its proper promoter was cloned into vectors pSB1 and pSB2 by amplifying it with the oligonucleotides MetBF and MetBR and cloning the PCR amplificate into the restriction sites PstI and HindIII.

MetBF (4125957-4125982) (SEQ ID NO 34):
ttagacagaa ctgcagcgcc gctccattca gccatgagat ac
MetBR (4127500-4127469) (SEQ ID NO 35):
cgtaacgccc aagcttaaat aacacttcac atcagccaga
ctactgcc

The resulting vectors are named pSB5 (pME101thrA*-metBEc) and pSB6 (pME101 thrA*-thrB-metBEc).

Subsequently the plasmids pSB3, pSB4, pSB5 and pSB6 were introduced into the MG1655 ΔmetJ metA*11 ΔthrC and MG1655 ΔmetJ metA*11 strains.

### EXAMPLE 4

### Production of Methionine Via Phosphohomoserine and/or O-Succinylhomoserine Using Sulfate or Hydrogen Sulfide as Sulfur Source

Amino acid production of the strains constructed in Examples 1 to 3 is analyzed in small Erlenmeyer flask cultures using modified M9 medium (Anderson, 1946, Proc. Natl. Acad. Sci. USA 32:120-128) that is supplemented with 5 g/l MOPS 5 g/l glucose and possibly 2 mM threonine. If hydrogensulfide is used as sulfur source all sulfate containing salts are replaced by equal molar amounts of chloride containing salts. Sulfide is supplied as ammonium sulfide (10 mM). Spectinomycin is added if necessary at a concentration of 100 mg/l. An overnight culture is used to inoculate a 30 ml culture to an OD600 of 0.2. After the culture has reached an OD600 of 4.5 to 5, 1.25 ml of a 50% glucose solution and 0.75 ml of a 2M MOPS (pH 6.9) are added and the culture is agitated for 1 hour. Subsequently IPTG is added if necessary.

Extracellular metabolites are analyzed during the batch phase. Amino acids are quantified by HPLC after OPA/Fmoc derivatization and other relevant metabolites are analyzed using GC-MS after silylation.

TABLE 2
Specific methionine production of strains harboring E. coli (pSB5) and
Arabidopsis (pSB3) MetB and their corresponding
Cystathionine-γ-synthase (CGS) and γ-elimination activities.

| | | | CGS | γ-elimination |
|---|---|---|---|---|
| | Methionine | Isoleucine | (mU/mg | (mU/mg |
| Strain | (mmol/gDw) | (mmol/gDw) | protein) | protein) |
| DmetBJ | 0.48 | 0.67 | 2468 | 307 |
| metA*11 pSB5 | | | | |
| DmetBJ | 0.26 | 0.13 | 6 | <0.05 |
| metA*11 pSB3 | | | | |

Production of methionine using METB from A. thaliana having low γ-eliminase activity significantly reduces the production of isoleucine through the γ-eliminase reaction, while methionine production is not strongly affected. This is in contrast to experiments in which exclusively the E. coli pathway is used and significant amounts of the by product isoleucine are produced via γ-elimination.

### EXAMPLE 5

### Construction and Evaluation of a Strain Producing Methionine Using the Yeast MetB Enzyme that Accepts Acetyl-Homoserine and H₂S as Substrates

To test the γ-eliminase activity of yeast in vivo and to evaluate its potential for the production of methionine, the following strain was constructed. The homoserine acetyl transferase encoding the MET2 gene of Saccharomyces cerevisiae was amplified by PCR from genomic DNA using the following oligonucleotides:

Ptac-metAlevF (SEQ ID NO 36):
tgctacagct ggagctgttg acaattaatc atcggctcgt
ataatgtgtg gaaggaggac agaccatgtc gcatacttta
aaatcgaaaa cgctccaaga gc
Ptac-metAlevR (SEQ ID NO 37):
CGTACTGACG ACCGGGTCCT ACCAGTTGGT AACTTCTTCG
GCCTCACC

Subsequently, the PCR fragment was cloned into the restriction sites PvuII and DrdI of the vector pACYC184, resulting in vector pSB7. The oligonucleotide Ptac-metAlevF harbors the pTAC promoter that drives the expression of the yeast MET2 gene from the vector pACYC184.

The yeast acetyl-homoserine sulfhydrylase (MET17) was amplified using the oligonucleotides metBlev and gapA-metBlevF. Simultaneously, the E. coli gapA promoter was amplified using the oligonucleotides gapA-metBlevR and GapAF. Subsequently the MET17 gene was fused to the gapA promoter by fusion PCR using only oligonucleotides metBlev and GapAF (see above for details).

gapA-metBlevR
(38-75:1860797-1860761; SEQ ID NO 38):
GGCCGGCGTG TAGTTGAACA GTATCGAAAT GAGATGGCAT
ATATTCCACC AGCTATTTGT TAGTGAATAA AAGG
gapA-metBlevF
(1-38:1860761-1860797; (SEQ ID NO 39):
ccttttattc actaacaaat agctggtgga atatatgcca
tctcatttcg atactgttca actacacgcc ggcc
metBlev (SEQ ID NO 40):
TAATCGCGGAT CCGCGTCATG GTTTTTGGCC AGCG
GapAF (1860639-1860661; SEQ ID NO 41):
acgtcccggg caagcccaaa ggaagagtga ggc

The fusion fragment was cloned into the SmaI and BamHI site of the vector pSB1 described in Example 1, resulting in vector pSB8.

Both vectors pSB7 and pSB8 were transformed into the strain ΔmetJ ΔmetB ΔmetA resulting in DmetBJ DmetC DmetA pSB7 pSB8. This strain grew after a lag period with a growth rate of 0.21 h−1. Plasmids and mutations were verified and the amount of methionine quantified.

TABLE 3
Specific methionine production of strains harboring E. coli (pSB5)
and yeast (pSB8) MetB and their corresponding O-succinyl
homoserine sulfhydrylase (OSHS), O-acetyl homoserine sulfhydrylase
(OAHS) and γ-elimination activities.

| | | | OSHS | |
|---|---|---|---|---|
| | | | or | |
| | | | OAHS | γ-elimination |
| | Methionine | Isoleucine | (mU/mg | (mU/mg |
| Strain | (mmol/gDw) | (mmol/gDw) | protein) | protein) |
| ΔmetBJ ΔmetC | 0.23 | 0.62 | 1704₁ | 307₁ |
| metA*11 pSB5 | | | | |
| ΔmetBJ ΔmetA | 0.57 | 0.14 | 194 | <0.05 |
| ΔmetC pSB7 pSB8 | | | | |

₁values were obtained in strain ΔmetBJ metA*11 pSB5.

Table 3 shows that the amount of methionine produced is significantly increased in the presence of the yeast MET2 and MET17 gene. At the same time the amount of isoleucine produced was reduced. This was explained by the low γ-eliminase activity of the MET17 enzyme (The γ-eliminase activity of the yeast acetyl-homoserine sulfhydrylase was determined in vitro as described in Example 1).

### EXAMPLE 6

### Construction and Evaluation of a Methionine Producing Strain Expressing Archaeal metB from Methanosarcina barkeri (Strain Fusaro)

As in plants, in archaea methionine biosynthesis is supposed to proceed via phosphohomoserine. Therefore, we presume that similar to the plant enzyme, MetB from Methanosarcina may also have a low γ-eliminase activity. Methanosarcina metB is therefore PCR amplified using the oligonucleotides metBmethano and gapA-metBmethanoR. As described previously, the gapA promoter from E. coli is subsequently fused to the metB gene using the oligonucleotides gapA-metBmethanoF and GapAF for the amplification of the promoter and metBmethano and GapAF for the actual fusion. The resulting fragment is cloned into the restriction site SmaI of the vector pJB137, resulting in plasmid pSB9.

MetBmethano (SEQ ID NO 42):
GTCCCCCGGG AATCTAGTCT AGATTAAATT ACTTCAAGG
GCCTGTTTGA GG
gapA-metBmethanoR
(32-69:1860797-1860761) (SEQ ID NO 43):
cacattttgt tgcaaacttc acttctcttt ccatatattc
caccagctat ttgttagtga ataaaagg
gapA-metBmethanoF
(1-38:1860761-1860797) (SEQ ID NO 44):
ccttttattc actaacaaat agctggtgga atatatggaaa
gagaagtgaa gtttgcaaca aaatgtg
GapAF (1860639-1860661) (SEQ ID NO 45):
acgtcccggg caagcccaaa ggaagagtga ggc

Plasmid pSB9 is introduced into strains ΔmetJ metA*11, ΔmetJ ΔmetA ΔmetB and ΔmetJ ΔmetA ΔmetB ΔthrC and the resulting strains are fermented as described in Example 2. Methionine production is increased and isoleucine production decreased when compared to the strain ΔmetJ metA*11 harboring the plasmid pSB5 with the E. coli metB gene. This is most likely due to the low γ-eliminase activity of the archaeal metB enzyme.

### EXAMPLE 7

### Construction and Evaluation of a Strain Expressing the metB Gene of Chloroflexus aurantiacus

The alignment in FIG. 2 shows that the MetB enzyme of Chloroflexus aurantiacus harbors several amino acids at positions required for the recognition of phosphohomoserine as a substrate. Therefore we presume that this enzyme like plant METB may have a lower γ-eliminase activity compared to the E. coli enzyme. To determine if the use of the metB enzyme confers an advantage in the production of methionine Chloroflexus metB is PCR amplified using the oligonucleotides metBchloro and gapA-metBchloroR. As described previously the gapA promoter from E. coli is subsequently fused to the metB gene using the oligonucleotides gapA-metBchloroF and GapAF for the amplification of the promoter and metBchloro and GapAF for the actual fusion. The resulting fragment is cloned into the restriction sites BamHI and SmaI of the vector pACYC177 (Biolabs), resulting in vector pSB10.

metBchloro (SEQ ID NO 46):
ACGTGGATCC GAATTCCTTA TTCGTCGGCA AGAGCCTGTT GC
gapA-metBchloroR
(33-70:1860797-1860761) (SEQ ID NO 47):
ggccgtacgg gtccggtaaa ctgatcgata gccatatatt
ccaccagcta tttgttagtg aataaaagg
gapA-metBchloroF
(1-38:1860761-1860797) (SEQ ID NO 48):
ccttttattc actaacaaat agctggtgga atatatggct
atcgatcagt ttaccggacc cgtacggcc
GapAF (1860639-1860661) (SEQ ID NO 49):
acgtcccggg caagcccaaa ggaagagtga ggc

To evaluate the enzyme in methionine biosynthesis the plasmid pSB10 is introduced into the strains ΔmetJ metA*11, ΔmetJ ΔmetA ΔmetB and ΔmetJ ΔmetA ΔmetB ΔthrC and the resulting strains are fermented as described in Example 2. Methionine production is significantly increased when compared to the strain ΔmetJ metA*11 harboring the plasmid pSB6 with the E. coli metB gene.

### EXAMPLE 8

### Construction of a Strain Expressing Yeast Homoserine Acetyltransferase and Acetylhomoserine Sulfhydrylase metY from Corynebacterium glutamicum

To test the γ-eliminase activity of C. glutamicum in vivo and to evaluate its potential for the production of methionine, the following strain is constructed.

The codon usage of the C. glutamicum acetyl-homoserine sulfhydrylase gene, metY, is adapted to E. coli and it is synthesized in vitro. Simultaneously the gapA promoter of E. coli is added.

The fusion fragment is subsequently cloned into the SmaI and BamHI site of the vector pSB1 described in Example 1, resulting in vector pSB11.

Both vector pSB7 carrying the yeast homoserine acetyltransferase and pSB11 are transformed into the strains MG1655 ΔmetJ metA*11 and ΔmetJ ΔmetB ΔmetA and the amount of methionine produced determined. It can be shown that the amount of methionine produced is significantly increased in the presence of the yeast homoserine acetyltransferase and the codon adapted Corynebacterial acetylhomoserine sulfhydrylase, metY, when grown with hydrogen sulfide or sulfate as sulfur source. At the same time, the amount of isoleucine produced is significantly reduced. Alternatively, a codon adapted homoserine transacetylase from Corynebacterium may be used instead of the yeast enzyme.

### EXAMPLE 9

### Construction of a Strain Expressing an E. coli Homoserine Succinyltransferase with Reduced γ-Elimination Activity

To assure that isoleucine is not produced via its classic biosynthesis pathway the gene ilvA and the operon tdcABCDEFG harboring a second threonine deaminase were deleted.

The ilvA gene was deleted using the deletion strategy described above and the following oligonucleotides:

DilvAF

(SEQ ID NO 50)

Ggctgactcgcaacccctgtccggtgctccggaaggtgccgaatatttaa
gagcagtgctgcgcgcgccggtttacgaggTGTAGGCTGGAGCTGCTTCG
with
a region (lower case) homologous to the sequence
(3952954-3953033) of the gene ilvA (reference
sequence on the website http://genolist.pasteur.
fr/Colibri/),
a region (upper case) for the amplification of the
chloramphenicol resistance cassette (reference
sequence in Datsenko, K.A. & Wanner, B.L., 2000,
PNAS, 97:6640-6645),
DilvAR

(SEQ ID NO 51)

cctgaacgccgggttattggtttcgtcgtggcaatcgtagcccagctcat
tcagccgggtttcgaaatccggttcatggCATATGAATATCCTCCTTAG
with
a region (lower case) homologous to the sequence
3954478-3954400) of the gene ilvA
a region (upper case) for the amplification of the
chloramphenicol resistance cassette.

The oligonucleotides DilvAR and DilvAF were used to amplify the chloramphenicol resistance cassette from the plasmid pKD3. The PCR product obtained was then introduced by electroporation into the strains MG1655 ΔmetBJ metA11 (pKD46) and ΔmetJ metA11 (pKD46) in which the Red recombinase enzyme expressed permitted the homologous recombination. The chloramphenicol resistant transformants were then selected and the insertion of the resistance cassette was verified by a PCR analysis with the oligonucleotides ilvAR and ilvAF defined below. The resulting strains was ΔmetBJ metA*11 ΔilvA.

ilvAR (3954693-3954670) (SEQ ID NO 52):
gccccgaaccggtgcgtaaccgcg
ilvAF (3952775-3952795) (SEQ ID NO 53):
ggtaagcgatgccgaactggc

In addition to IlvA, the threonine dehydratase (TdcB) is known to catalyze the deamination of threonine to α-ketobutyrate under anaerobic or microaerobic conditions. To eliminate the possible contribution of this enzyme to α-ketobutyrate production, the gene was deleted from the genome of the strain ΔmetBJ metA*11 ΔilvA. TdcB is part of the operon tdcABCDEFG that was deleted in a similar way as described for previous mutants using the four oligonucleotides described below. DtdcGR and DtdcAF were used to amplify the cassette and tdcGR and tdcGF for the verification

DtdcGR (3255915-3255993)

(SEQ ID NO 54)

gctgacagcaatgtcagccgcagaccactttaatggccagtcctccgcgt
gatgtttcgcggtatttatcgttcatatcCATATGAATATCCTCCTTAG
DtdcAF (3264726-3264648)

(SEQ ID NO 55)

Ggtaattaacgtaggtcgttatgagcactattcttcttccgaaaacgcag
cacctggtagtctttcaggaagtcattagTGTAGGCTGGAGCTGCTTCG
tdcGR (3255616-3255640)

(SEQ ID NO 56)

gcgtctgcaatgacgcctttattcg
tdcAF (3264922-3264899)

(SEQ ID NO 57)

Cgccataaaatatggttatccccg

The resulting strain was called ΔmetBJ metA*11 ΔilvA ΔtdcABCDEFG.

To decrease the γ-eliminase activity of E. coli, cystathionine-γ-synthase/sulfhydrylase (MetB) mutations were introduced into regions that are involved in the binding of the substrate cysteine. To this end, Escherichia coli metB was PCR-amplified from genomic DNA using the oligonucleotides MetBF and MetBR (numbers in parentheses correspond to positions on the E. coli genome). The PCR fragment was restricted by PstI and HindIII and cloned into pUC18 into the same restriction sites.

MetBF (4125957-4125982) (SEQ ID NO 58):
ttagacagaa ctgcagcgcc gctccattca gccatgagat ac
MetBR (4127500-4127469) (SEQ ID NO 59):
cgtaacgccca agcttaaata acacttcaca tcagccagac
tactgcc

Subsequently, mutations were introduced into the Escherichia coli cystathionine-γ-synthase/sulfhydrylase that result in the amino acid changes T335A/A337P, R49L and D45V.

The following pairs of oligonucleotides were used for the introduction of each mutation using site directed mutagenesis according to Stratagene's Quick Change™ site directed mutagenesis KIT. Restriction sites (bold) were introduced for verification.

metBT335A/A337PF (SEQ ID NO 60):
cgcgcttctg gtgccatgcc cggatgtgcc atggttgcgg cg
metBT335A/A337PR (SEQ ID NO 61):
cgccgcaacc atggcacatc cgggcatggc accagaagcg cg
metBR49LF (SEQ ID NO 62):
gaacctcgagcgcatgattactcgcgtctgggcaacccaacgcgcg
metBR49LR (SEQ ID NO 63):
cgcgcgttgggttgcccagacgcgagtaatcatgcgctcgaggttc
metBD45VF (SEQ ID NO 64):
gaacctcgagcgcatgtgtactcgcgtcgcggcaacccaacgcgcgat
metBD45VR (SEQ ID NO 65):
atcgcgcgttgggttgccgcgacgcgagtacacatgcgctcgaggttc

The resulting modified metB sequences were verified by sequencing, restricted with PstI and HindIII and cloned into the same sites of vector pSB1. The resulting plasmids were transformed into the strain ΔmetBJ metA*11 ΔilvA ΔtdcABCDEFG. Strains were evaluated in small Erlenmeyer flask cultures as described above. The amount of isoleucine produced by γ-elimination was significantly reduced, whereas methionine synthesis was only slightly affected. This correlates with the low γ-elimination activity and the retention of a significant cystathionine-γ-synthase activity.

| | | Iso | CGS | γ-elim |
|---|---|---|---|---|
| | Meth | (mmol/ | (mU/mg | (mU/mg |
| Strain | (mmol/gDw) | gDw) | protein) | protein) |
| DmetBJ metA*11 DilvA | 0.96 | 0.88 | 3790 | 101 |
| DtdcABCDEFG pSB5 | | | | |
| DmetBJ metA*11 DilvA | 0.24 | 0 | 425 | 1 |
| DtdcABCDEFG pSB1- | | | | |
| metB**T335A/A337P | | | | |
| DmetBJ metA*11 DilvA | 0.69 | 0.33 | 891 | 34 |
| DtdcABCDEFG pSB1- | | | | |
| metB**D45V | | | | |
| DmetBJ metA*11 DilvA | 0.99 | 0.48 | 445 | 22 |
| DtdcABCDEFG pSB1- | | | | |
| metB**R49L | | | | |

1-26. (canceled)

27. A method of preparing an amino acid, its precursors or derivatives, comprising:
a) fermenting a microorganism producing the amino acid
b) concentrating the amino acid in cells of bacteria or in a medium and
c) isolating the desired amino acid/constituents of a fermentation broth and/or a biomass optionally remaining in portions or in a total amount (0-100%) in an end product,
wherein said microorganism expresses enzymes that have one or several of the following activities: cystathionine-γ-synthases and/or phosphohomoserine sulfhydrylase and/or acylhomoserine sulfhydrylases, and that have at the same time a γ-elimination activity that is at least two times inferior when compared to the E. coli cystathionine-γ-synthase.

28. The method according to claim 27, wherein said enzymes have a γ-elimination activity that is at least 10 times inferior when compared to E. coli cystathionine-γ-synthase.

29. The method of claim 27, wherein said microorganism expresses phosphohomoserine accepting cystathionine-γ-synthase/phosphohomoserine sulfhydrylase.

30. The method of claim 27, wherein said microorganism expresses cystathionine-γ-synthase and/or phosphohomoserine sulfhydrylase encoded by the METB gene of plants

31. The method of claim 30, wherein the metB gene is from Arabidopsis thaliana.

32. The method of claim 27, wherein said microorganism expresses cystathionine-γ-synthase and/or O-acetyl homoserine sulfhydrylase encoded by the METB gene of Saccharomyces cerevisiae.

33. The method of claim 27, wherein said microorganism expresses cystathionine-γ-synthase and/or phosphohomoserine sulfhydrylase that is derived from Methanosarcina barkeri.

34. The method of claim 27, wherein said microorganism expresses cystathionine-γ-synthase and/or phosphohomoserine sulfhydrylase that is derived from Chloroflexus aurantiacus.

35. The method of claim 27, wherein said microorganism expresses an enzyme that have one or several of the following activities: cystathionine-γ-synthases and/or phosphohomoserine sulfhydrylase and/or acylhomoserine sulfhydrylases, and that have at the same time a γ-elimination activity that is at least two times inferior when compared to E. coli cystathionine-γ-synthase, said enzyme having at least two of the following amino acids at positions 107E, 111Y, 165K, 403S.

36. The method of claim 27, wherein said microorganism expresses cystathionine-γ-synthase and/or acylhomoserine sulfhydrylase encoded by the metY and/or the metB gene of gram-positive bacteria.

37. The method of claim 27, wherein said microorganism expresses native or heterologous cystathionine-γ-synthase and/or phosphohomoserine sulfhydrylase and/or acylhomoserine sulfhydrylase enzyme that is optimized and as a consequence has a lower γ-eliminase activity and thus allows increased methionine selectivity at the expense of isoleucine.

38. The method of claim 27, wherein said microorganism expresses an optimized cystathionine-γ-synthase and/or phosphohomoserine sulfhydrylase and/or acylhomoserine sulfhydrylase enzyme that has a proline at position 337 and/or an alanine at position 335.

39. The method of claim 27, wherein said microorganism overexpresses a polynucleotide which codes for cystathionine-γ-synthase and/or phosphohomoserine sulfhydrylase and/or acylhomoserine sulfhydrylase.

40. The method of claim 27, wherein said microorganism expresses cystathionine-γ-synthase and/or phosphohomoserine sulfhydrylase and/or acylhomoserine sulfhydrylase whose catalytic properties are improved.

41. The method of claim 27, wherein heterologous homoserine acyltransferases are introduced into the microorganism, that permit the use of a corresponding acylhomoserine accepting cystathionine-γ-synthase and/or acylhomoserine sulfhydrylases.

42. The method of claim 27, wherein several different hybrid pathways are actively producing homocysteine and/or cystathionine from homoserine.

43. The method of claim 27, wherein further genes of a biosynthesis pathway of the amino acid to be produced are additionally enhanced.

44. The method of claim 27, wherein metabolic pathways that reduce production of the amino acid are at least partially reduced.

45. The method of claim 27, wherein a sulfur molecule/compound is transferred to any activated homoserine from cysteine.

46. The method of claim 27, wherein a sulfur molecule/compound is transferred directly from H₂S to any activated homoserine.

47. The method of claim 27, wherein a sulfur source in the medium is sulfate or a derivative.

48. The method of claim 27, wherein a sulfur source in the medium is thiosulfate.

49. The method of claim 27, wherein a sulfur source in the medium is H₂S.

50. The method of claim 27, wherein a sulfur source in the medium is methylmercaptan.

51. A microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzymes are derived from one of the following species: Saccharomyces cerevisiae, Methanosarcina barkeri, and Chloroflexus aurantiacus.

52. A microorganism that expresses an enzyme having one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzyme has at least two of the following amino acids at positions 107E, 111Y, 165K, 403S.

53. A microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzymes are encoded by one of the following genes: metB from Saccharomyces cerevisiae, metY and/or metB from gram-positive bacteria.

54. A microorganism in which enzymes are expressed that have one or several of the following activities: cystathionine-γ-synthases and/or acylhomoserine sulfhydrylases, and that have at the same time a γ-elimination activity that is at least two times inferior when compared to the E. coli cystathionine-γ-synthase, wherein said enzyme has a proline at position 337 and/or an alanine at position 335.
